(19) United States
(12) Patent Application Publication
(10) Pub. No.: US 2004/0175782 A1
(43) Pub. Date: Sep. 9, 2004

(54) Biotransformation of compounds using non-prokaryotic microalgae

(75) Inventor: Nancy G. Kravit, Bangor, ME (US)

Correspondence Address: SUGHRUE MION, PLLC, 2100 PENNSYLVANIA AVENUE, N.W., SUITE 800, WASHINGTON, DC, 20037, US

(73) Assignee: TETHYS RESEARCH LLC

(21) Appl. No.: 10/379,939

(22) Filed: Mar. 6, 2003

(51) Int. Cl. C12P 1/00; C12P 17/14

(52) U.S. Cl. 435/41

(57) ABSTRACT

A method for biotransformation of organic compounds using non-prokaryotic microalgae is disclosed. The method is useful to biotransform a chemical precursor compound, preferably a heterocyclic compound, to a chemically distinct final product, which is useful in, e.g., pharmaceutical, agrichemical, nutraceutical, ecological, hazardous waste, food flavoring, or food additive applications.

### FIELD OF THE INVENTION

The present invention relates to a method for biotransformation of compounds using non-prokaryotic microalgae so as to produce metabolites which are useful in, e.g., pharmaceutical, agrichemical, nutraceutical, ecological, food flavoring, or food additive applications.

### BACKGROUND OF THE INVENTION

Biotransformation is the strategy of using living organisms to perform chemical reactions that are difficult for chemists to accomplish in the laboratory or that are desired to yield a product that is functionally active in another living system. Single or multiple precursor molecules are provided to the living system, and after time is allowed for metabolism to occur, a product or products, consisting of a single or a small number of enzymatic modifications of the precursor molecule(s), are isolated from the medium or the biomass. One of the first commercial processes to use a biotransformation step was the hydroxylation of a C₂₁ steroid by Rhizopus in 1952 (Murray et al, U.S. Pat. No. 2,602,769). Such an approach has proven its utility for the bioconversion of one molecule into another over a more than fifty year history (e.g., Bombardelli et al, U.S. Pat. No. 6,372,458; Burns et al, U.S. Pat. No. 6,361,979; Chartrain et al, U.S. Pat. No. 5,849,568; Hou et al, U.S. Pat. No. 5,852,196; Lesage-Messen et al, U.S. Pat. No. 5,866,380 and Takashima et al, U.S. Pat. No. 6,365,399).

As can be seen in the aforementioned references, the organism of choice for biotransformation has traditionally been a bacteria or a fungus. The reasons for this are straightforward. Since the full extant and capability of even the simplest organism's biochemistry was (and still is except in a few cases) unknown, the choice of a test organism was dictated by concerns for diversity and ease of culture. Bacteria and fungi are usually very easy to cultivate in the laboratory, they can grow as pure cultures (axenic growth), divide rapidly, and be cultivated using a simple medium of defined composition that makes subsequent chemical manipulation simpler and therefore less costly. Bacteria and fungi are also evolutionarily and ecologically diverse. In fact, conventional wisdom in biotransformation is that since all environments have bacteria and most have fungi, members of these two groups will have encountered all of the naturally occurring organic compounds and developed enzymatic machinery to make, modify, or degrade them.

There are more than 137 functional organic chemical groups found in nature (University of Minnesota Biocatalysis/Biodegradation Database (UM-BBD), http://umbbd.ahc.umn.edu; and Wackett et al, “Biocatalysis and Biodegradation”, Microbial Transformation of Organic Compounds, ASM Press, Washington, D.C. (2001)). While some of these are the result of non-biological processes, such as lightening or fire, most of these functional groups were produced by microorganisms. Consequently, enzymes must have created those functional groups. In addition, organisms living in the environment encounter the chemical groups produced by enzymes as well as those produced by physical processes like fire. As a result, there is a high probability that at least some organisms have developed the enzymatic machinery to degrade both the biologically and physically produced groups. Currently, about 40% of the known natural functional organic chemical groups are known to be degraded by bacteria and fungi.

The increasing reliance of industry on biotechnology has led to an increased effort to find useful enzymes in bacteria and fungi. However, as shown in FIG. 1, a survey of biotransformation patents granted has shown that fewer biotransformation patents are being granted. The trend is even more striking when viewed against the background of an increasing number of patents granted in the patent categories including biotransformation. The decreasing number of biotransformation patents in an era of expanding numbers of biotechnology patents suggests a need for a novel approach. This conclusion is supported by the number of new strategies being applied to biotransformation. For example, nearly complete information is available on the metabolism of a few simple bacteria and yeast, such as Esherchia coli and Saccharomyces cerviserii. Some researchers are engineering the metabolism of bacteria or yeast to increase the efficiency of biotransformation, or they are transforming a variety of cells with genes from other organisms in order to accomplish specific biotransformations (Grabley et al, “Drug Discovery from Nature, eds., Springer-Verlag, Berlin (1999); and Ward et al, Critical Reviews in Biotechnology, 18:25-83 (1998)). However, metabolic and genetic engineering is extremely time-consuming and expensive compared with traditional biotransformation strategy. In addition, some companies (e.g., Diversa Corp.) have used environmental DNA libraries for enzyme discovery.

Such libraries are generally limited to organisms whose genes do not contain introns (mostly bacteria) and require probing with oligonucleotides, and so they are best suited to finding variants on known enzymes as opposed to the discovery of new ones.

Other groups have taken advantage of the fact that under ordinary culture conditions, only a subset of a cell's potential metabolic reactions occur. The cell expresses certain enzymes only under certain environmental conditions. The textbook example of such metabolic pathways is the lac operon of Escherichia coli. The bacteria do not make the enzyme that modifies β-galactosides unless glucose is absent and lactose (or a closely related molecule) is present. So one approach to increasing the potential of biotransformation is to grow each of the microorganisms used in a given experiment under a variety of differing culture conditions (Grabley et al, supra; and Sattler et al, In: Drug Discovery from Nature, eds. Grabley et al, Springer-Verlag, Berlin, pages 191-214 (1999)). This approach vastly increases the number of cultures to be screened, and so it also is expensive compared to the traditional strategy of biotransformation.

Another strategy to increase the number of biochemical reactions available for biotransformation is to increase the diversity of the population of organisms used for biotransformation. For example, cultured cells from various organs of multicellular organisms like mammals and plants have been used for biotransformations (Balani et al, U.S. Pat. No. 5,387,512; Labuda et al, U.S. Pat. No. 5,279,950; and Mangold, Chemistry and Industry, 8:260-267 (1989)). While these approaches have yielded a number of useful results, the culture of cells of higher multicellular organisms is very expensive in terms of both equipment and media. In addition, such cells usually require complex media that makes the subsequent analysis for modified precursor molecules far more difficult, and hence very costly.

In sum, new strategies for biotransformation using bacteria and fungi are more costly than traditional methods and have not sufficiently increased the range of useful reactions. Thus, there is a need for a novel approach to efficiently and at moderate to low cost, increase the range of enzyme reactions available to biotechnology. The present invention provides a simple and cost-effective way to increase the utility and range of biotransformation by using non-prokaryotic microalgae (hereinafter referred to as “microalgae”), to transform precursor molecules. As discussed above, the use of microorganisms other than bacteria and fungi for biotransformation runs counter to established thought in the art.

Microalgae, as discussed below, are diverse evolutionarily, metabolically, and ecologically. In addition, many microalgae can be grown in a defined simple medium that simplifies subsequent isolation and permits identification of the modified precursor with less cost. The microalgae are an extraordinarily diverse group of organisms with a polyphyletic origin. The taxonomy of the microalgae is not yet definitively determined, but authorities place the origins of the microalgae into 12-14 phyla in as many as 4 kingdoms (Graham et al, Algae, Prince Hall, NJ (2000); Saunders et al, Proc. Natl. Acad. Sci., USA, 92:244-248 (1995); and Wainright et al, Science, 260:340-342 (1993)). For comparison, the fungi are usually grouped into only one kingdom. Further proof of the diversity of microalagae comes from their cellular characteristics. They can be uninucleate, coenocytic or siphonous. Some groups of microalgae are true eukaryotes while others are mesokaryotes (Bold et al, “Introduction to Algae”, Structure and Reproduction, Prentice-Hall, Inc., Englewood Cliffs, N.J. (1985)), an evolutionary offshoot on the pathway from prokaryotes toward eukaryotes. Mesokaryotes, for example, lack the chromosomal histones of true eukaryotes.

In addition to their evolutionary diversity, the microalgae exhibit a wide range of ecological diversity. Species live in marine, freshwater and soil habitats. There are microalgae in some of the most extreme environments on earth, such as the Great Salt Lake (Lee, Phycology, 2^{nd} edn., Cambridge University Press, Cambridge UK (1989)), deserts, and even inside rocks (Friedmann et al, Microbial Ecol., 16:271-289 (1988)). Some species are autotrophic, using light and CO₂ for their source of energy and organic carbon, while others are heterotrophic, metabolizing organic carbon compounds from the environment for both energy and synthetic pathways. Either growth mode can be obligate, or the growth mode can be facultative, switching on only when needed. Some algae are even mixotrophic, photosynthesizing while supplementing carbon fixation by heterotrophy (Graham et al, supra).

Further proof of the wide diversity in algal metabolism also comes from their production of compounds not synthesized in other taxonomic groups. For example, some algae synthesize unusual long-chain polyunsaturated fatty acids like eicosapentaenoic aicd (Pohl, In: Zaborsky, ed. Handbook of Biosolar Resources, CRC Press, Boca Raton, Fla., pages 383-404 (1982); and Shimoda et al, J. Molecular Catalysis b: Enzymatic, 8:255-264 (2000)). Others synthesize uncommon storage molecules like paramylon and chrysolaminaran (Graham et al, supra) and unusual sugar molecules, both mono- and polysaccharides (O'Colla, In: Lewin, R. A. (ed.), Physiology and Biochemistry of Algae, Academic Press, NY, pages 337-356 (1962)). The unusual sugars are particularly noteworthy, since molecules containing sugar groups are becoming increasingly important in pharmaceuticals (Grabley et al, supra; and Sattler et al, supra).

There have been some previous attempts to harness the variety of biochemical reactions present in microalgae. In particular, various groups have focussed on substituted aromatic aldehydes. The aromatic aldehydes, especially those with three or more fused rings, comprise a class of hazardous environmental pollutants accumulating mostly because of fossil fuel combustion (Cerniglia, Biodegradation, 3:351-368 (1992)) and are generally refractory to biodegradation by bacteria or fungi. The initial step towards the development of a biodegradation system for substituted aromatic aldehydes is the degradation of unsubstituted aromatic aldehydes. Degradation of naphthalene to 1-naphthol by microalgae was first shown in the late 1970's (Cerniglia et al, Appl. Env. Microb., 34:363-370 (1977); Cerniglia et al, J. Gen. Microb., 116:495-500 (1980a); Soto et al, Canadian J. Microb., 53:109-117 (1975a); Soto et al, Candian J. Microb., 53:118-126 (1975b); and Winters et al, Marine Biol., 36:269-276 (1976)). A number of related aldehydes have since been shown to be reduced to the corresponding alcohol (i.e., Cerniglia, Biodegradation, 3:351-368 (1992); Cerniglia et al (1980a), supra; Cerniglia et al, Arch. Microb., 125:203-207 (1980b); Hook et al, Phytochemistry, 51:621-627 (1999); Noma et al, Phytochemistry, 31:515-517 (1992a); Noma et al, Phytochemistry, 31:2009-2011 (1992b); Noma et al, Phytochemistry, 30:1147-1151 (1990); Noma et al, Phytochemistry, 30:2969-2972 (1991); Shimoda et al, supra; and Warshawsky et al, Chemico-biological Interactions, 97:131-148 (1995)). Kniefel and co-workers have shown that the substituted aromatic 1-napthalenesulfonic acid can be transformed by Scenedesmus to 1-hydroxy-2-napthalenesulfonic acid (Kneifel et al, Arch. Microbiol., 167:32-37 (1997)). Gutenkauf and co-workers have investigated the biotransformation of 4-chloro-3,5-dinotrobenzoic acid in Chlamydomonas and found that it was partially converted into 3,5-dinitro-4-hydroxybenzoic acid (Gutenkauf et al, Biodegradation, 9:2359-2368 (1998)).

In 1986, Abul-Hajj and Qian looked at the ability of microalgae to reduce unsubstituted aromatic aldehydes and reasoned that those steroids that are also aromatic aldehydes might also be reduced by microalgae (Abul-Hajj et al, J. Nat. Products, 49:244-248 (1986)). They were able to demonstrate that some algae could modify 4-androstene-3,17-dione and 17-β-hydroxy-4-androstene-3-one by reducing the oxygen at the 17 position or hydroxylating the steroid at the 6-β or 14-α positions. This work has been further explored by an Italian group (Della Greca et al, Phytochemistry, 41:1527-1529 (1996); Della Greca et al, Tetrahedron, 53:8273 (1997); Greca et al, Biotechnology Letters, 19:1123-1124 (1997); Greca et al, Biotechnology Letters, 18:639-642 (1996); Pollio et al, Phytochemistry, 37:1269-1272 (1994); and Pollio et al, Phytochemistry, 37:1269-1272 (1994)), as well as a commercial group in the United States, culminating in the award of a patent for the production of a testosterone derivative that is an inhibitor of testosterone 5-α reductase (Arison et al, U.S. Pat. No. 5,215,894).

Some algae have also been used for other very specific biotransformations. For example, Chlorella has been used to convert cyclohexaneacetic acid to monohydroxyclohexaneacetic acid (Yoshizako et al, J. Fermentation and Bioengineering, 72:343-346 (1991)). Similarly, Selenastrum capricornutum has been used to biotransform finasteride, another inhibitor of testosterone 5-α reductase (Venkataramani et al, Ann. N.Y. Acad. Sci., 745:51-60 (1994)). This transformation is particularly interesting because it introduces a hydroxy group not by converting a previous aldehyde carbonyl but, rather by displacing a hydrogen atom. In addition to the reduction of aromatic aldehydes, microalgae have been used for the production of novel long-chain polyunsaturated fatty acids (Certik et al, J. Fermentation and Bioengineering, 87:1-14 (1999); and Fauconnot et al, Phytochemistry, 47:1465-1471 (1998)).

Thus, microalgae provide a useful reservoir of unexplored biochemical reactions (Radmer et al, J. Appl. Phycology, 6:93-98 (1994)). Nevertheless, the power of microalgae has not been well exploited in general, and specifically, the power of microalgae for biotransformation has not been appreciated. The general strategy of using microalgae to perform steps in a chemical synthesis has been to look at a known specific chemical reaction in a specific alga and to see if the alga can transform a closely-related substrate (i.e., Della Greca et al (1996), supra; Della Greca et al (1997), supra; Fauconnot et al, supra; Greca et al (1997), supra; Greca et al (1996), supra; Kneifel et al, supra; Noma et al (1992a), supra; Noma et al (1992b), supra; Noma et al (1990), supra; Pollio et al (1996), supra; and Shimoda et al, supra). A few studies have employed panels of microalgae (Abul-Hajj et al, supra; Cerniglia et al (1980a), supra; Hook et al, supra; and Pollio et al (1994), supra), but these studies have always looked for variant on the conversion of an aromatic aldehyde into an aromatic alcohol, a biochemical reaction previously known to be widely distributed in algae (Cerniglia et al (1997), supra; Soto et al (1975a); Soto et al (1975b), supra; and Winters et al, supra).

It is clear from the foregoing that, while microorganisms, including microalgae, can and have been used for the production of new chemical entities, microalgae have not been fully explored. While they have been investigated for natural products or for known enzyme activities, no studies to date have reported the systematic application of microalgal panels for discovery and development of novel intermediates in the production of high value chemical entities.

FIG. 2 shows some of the naturally occurring compounds that are not substrates for any known enzymes. Naturally occurring compounds that have so far been refractory to enzymatic conversion include many types of compounds, including heterocyclic and non-heterocyclic compounds. In the past 50 years, much effort has been expended in trying to find enzymes in bacteria and fungi that can modify these molecules. Despite the argument that some bacteria and fungi will have encountered these compounds in the environment and will have evolved some means of catabolizing them, no such organisms have been discovered at the current time. It has been found in the present invention that microalgae contain a large untapped reservoir of potential enzymes for modifying or degrading organic molecules. Because microalgae synthesize organic compounds not found in other organisms (i.e., O'Colla et al, supra), they are also believed in the present invention to be a valuable resource for enzymes to help synthesize complicated chiral pharmaceuticals. Since microalgae also synthesize unusual polymers (Graham et al, supra; Pohl, supra; and Shimoda et al, supra), they are further believed in the present invention to be important sources of enzymes to degrade or modify molecules with long carbon backbones, like petroleum products or some kinds of plastics. Discovery of unique microalgal enzymes requires a method to systematically and efficiently screen microalgal panels. Thus, a method involving the use of microalgal panels to biotransformation would prove critical for production of pharmaceuticals and other high value products in a manner which is currently not possible.

### SUMMARY OF THE INVENTION

To date, biotransformations in algae have looked for limited, predetermined reactions and products. The present invention takes a different and unique approach of systematically harnessing randomness. The process uses panels (series) of microalgae to biotransform a known chemical compound into a new chemical compound that was not predetermined. The new chemical compounds are subsequently identified and examined for potential applications. The principal advantage of the present invention is that panels of microalgae can be screened for the performance of biotransformation reactions efficiently and cost-effectively, as well as over a wider range of biotransformations than available through the use of biotranformation systems comprised of bacteria and fungi. Additional features and advantages of the invention will be set forth in the description which follows, and will be apparent from the description, or may be learned by practice of the invention.

To achieve these and other advantages and in accordance with the purpose of the invention, as embodied and broadly described, the present invention is in part based on the discovery that panels of microalgae can biotransform chiral and non-chiral organic compounds very efficiently and effectively. The present invention provides a process for the biotransformation, in a microalgal organism, of a chemical precursor compound to a chemically distinct final product, useful in, e.g., pharmaceutical, agrichemical, nutraceutical, ecological, food flavoring, and food additive applications. The present invention also provides a process for the biotransformation, in a microalgal organism, of a racemic mixture of a chemical precursor compound into a more chirally pure state, useful in, e.g., pharmaceutical, agrichemical, nutraceutical, ecological, food flavoring, and food additive applications.

In one embodiment, the invention relates to a process for biotransformation of a precursor compound comprising:

(A) obtaining a panel of different non-prokaryotic microalgae which are evolutionarily and ecologically diverse;

(B) exposing each member of said panel of non-prokaryotic microalgae to a solvent for the precursor compound, and selecting a subset of non-prokaryotic microalgae that grow in the presence of said solvent;

(C) exposing each non-prokaryotic microalgae of the resulting subset to the precursor compound, and selecting a further subset of non-prokaryotic microalgae whose growth is inhibited or increased in the presence of said precursor compound;

(D) growing the resulting further subset of non-prokaryotic microalgae in the presence of said precursor compound so as to transform said precursor compound to produce a metabolite of said precursor compound, and so as to obtain a cellular biomass of said non-prokaryotic microalgae and a culture supernatant;

(E) separating the resulting cellular biomass from the resulting culture supernatant and optionally extracting the separated cell biomass with said solvent so as to obtain said metabolite of said precursor compound; and optionally

(F) purifying said metabolite from the resulting culture supernatant or solvent extract of the resultant biomass and analyzing said metabolite so as to identify the structure of said metabolite and the modification in said precursor compound.

The present invention further comprises a process for obtaining said metabolite by culturing a member of said further subset of non-prokaryotic microalgae in the presence of said precursor compound, and purifying the resulting metabolite from the resulting culture.

The present invention still further comprises a metabolite obtainable by said process.

The present invention also provides for a method comprising contacting a mammal with the metabolite so obtained, and assaying for toxicity of said metabolite in said mammal, wherein said precursor compound is a pharmaceutical, a food additive or a hazardous waste.

### BRIEF DESCRIPTION OF THE DRAWINGS

FIG. 1 shows trends in biotransformation patents granted during recent 5 year periods. The database of the United States Patent and Trademark Office was searched for patents in Class 435, subclasses 43-67 for biotransformation patents, as well as for all patents in those subclasses. The results were corrected for those patents appearing in more than one subcategory. The biotransformation patents were manually examined to eliminate irrelevant results, such as those patents concerned with apparatus design.

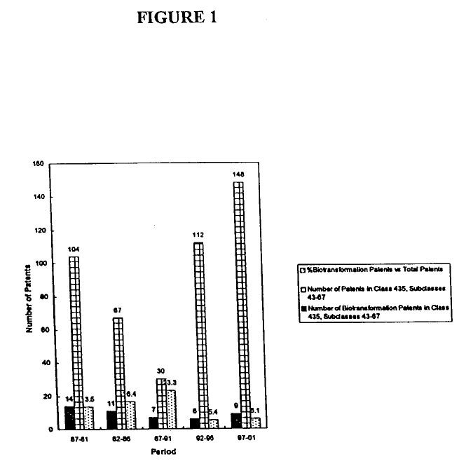

FIG. 2 shows examples of chemical groups found in nature that are not known to be modified by bacteria and fungi.

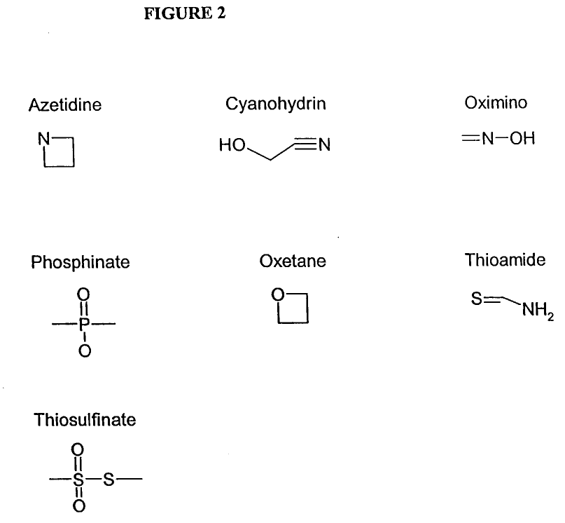

FIG. 3 shows the growth (cell counts) of various microalgae grown in the presence of 100 μg/ml of (S)-(−)-3-(Benzyloxycarbonyl)-4-oxazolidinecarboxylic acid over the course of time.

FIGS. 4A-4B show HPLC analyses of solvent extracts of culture supernatants of Chlamydomonas reinhardtii incubated with and without 100 μg/ml of (S)-(−)-3-(Benzyloxycarbonyl)-4-oxazolidinecarboxylic acid. FIG. 4A shows the HPLC results of experimental and control samples. A new peak present in the experimental, but not in control culture supernatants is marked. FIG. 4B shows the area of the chromatogram around the marked peak in FIG. 4A (shown at increased resolution). In addition, in FIG. 4B, HPLC analyses of culture medium that had not been incubated with cells is shown.

FIGS. 5A-5D show LC/MS and LC/MS/MS of (S)-(−)-3-(Benzyloxycarbonyl)-4-oxazolidinecarboxylic acid (precursor compound) and the material from the HPLC peak marked in FIG. 4B. FIG. 5A shows LC/MS of the precursor compound. FIG. 5B shows LC/MS of the material from the peak marked in FIG. 4A. FIG. 5C shows LC/MS/MS of the precursor compound. FIG. 5D shows LC/MS/MS of the material from the peak marked in FIG. 4B.

FIGS. 6A-6B shows the structure of the precursor compound, (S)-(−)-3-(Benzyloxycarbonyl)-4-oxazolidinecarboxylic acid (FIG. 6A), and the metabolite thereof, i.e., (S)-(−)-3-(Benzyloxycarbonyl)-1-amino-2-hydroxycarboxylic acid (FIG. 6B).

FIG. 7 shows the growth (cell counts) of various microalgae grown in the presence of 100 μg/ml of tert-butyl[S-(R*-R*)]-(−)-(1-oxiranyl-2-phenylethylcarbamate) over the course of time.

FIGS. 8A-8B show LC (FIG. 8A) and MS (FIG. 8B) of auto-degradation of tert-butyl[S-(R*-R*)]-(−)-(1-oxiranyl-2-phenylethylcarbamate).

FIGS. 9A-9B show HPLC analysis of solvent extracts of culture supernatants of Cryptomonas ovata incubated with or without 100 μg/ml of tert-butyl[S-(R*-R*)]-(−)-(1-(oxiranyl-2-phenylethylcarbamate). FIG. 9A shows the HPLC results of experimental and control samples. A new peak present in the experimental, but not in the control culture supernatants is marked. FIG. 9B shows the area of the chromatogram around the marked peak in FIG. 9A (shown at increased resolution). In addition, in FIG. 9B, the HPLC analyses of culture medium that had not been incubated with cells is shown.

FIGS. 10A-10B show LC/MS and LC/MS/MS of tert-butyl[S-(R*-R*)]-(−)-(1-(oxiranyl-2-phenylethylcarbamate) (precursor compound). FIG. 10A shows the LS/MS of the precursor compound; and FIG. 10B shows the LC/MS/MS of the precursor compound.

FIG. 11 shows the proposed fragmentation pattern of tert-butyl[S-(R*-R*)]-(−)-(1-(oxiranyl-2-phenylethylcarbamate).

FIG. 12 shows the LC/MS of the metabolite of tert-butyl[S-(R*-R*)]-(−)-(1-(oxiranyl-2-phenylethylcarbamate).

FIG. 13 shows the LC/TS/MS of the m/z 385 metabolite.

FIG. 14 shows the proposed fragmentation pattern of the hypothetical metabolite, N¹-(tert-butoxycarbonyl)-N¹-[1-phenyl(2,3-dihydroxypropyl)methyl]cysteinamide.

FIG. 15 shows the proposed fragmentation pattern of the alternative hypothetical metabolite S-{2-hydroxy-3-[(tert-butoxycarbonyl)amino]-4-phenylbutyl}cysteine.

### THE DETAILED DESCRIPTION OF THE INVENTION

The term “biotransformation” is used herein to mean the process of exposing a microorganism, specifically a non-prokaryotic microalgae, to a precursor compound, where the compound is not considered to be a normal microalgae growth substrate, and identifying, after growth of the non-prokaryotic microalgae, a product that is a modification of the precursor compound, i.e., a metabolite, which results from the action of a single or very few enzymes on the precursor compound. The metabolite may be a chirally pure form of a racemic precursor compound.

The size of the initial panel of microalgae is not crtitical to the present invention. For example, the panel may contain 7 to 50 different microalgae strains, preferably 15 to 30 different microalgae strains.

A. Non-Prokaryotic Microalgae Strains

The particular species of non-prokaryotic algae chosen for the panel are not critical to the nature of the invention. Microalgal candidates may be chosen according to considerations of evolutionary diversity and ecological diversity. For example, candidates can be chosen to represent a wide variety of microalgal phyla. One example might be to chose at least one candidate each from phyla Charophyta, Chlorarachniophyta, Chlorophyta, Cryptophyta, Diatoms, Euglenophyta, Haptophyta, Heterokonta, and Rhodophyta. The ecological diversity is also preferably maximized. Thus, the algal strains chosen include, but are not limited to, freshwater benthic, epiphytic, and planktonic species (inhabiting for example flowing sources, alkaline creeks, eutrophic lakes and ponds, oligotrophic lakes and ponds, or alpine lakes and ponds), marine algae (including tropic, temperate and cold water variants of benthic, epiphytic and planktonic habitats, living in near shore, in open ocean, in brackish water or in halophilic environments), and non-aquatic microalgae such as antarctic algae, desert-dwelling algae, and soil algae, as well as species of unusual habitat such as ecotoparasitic algae and extremophilic algae from hot springs, snow and other extreme environments. Other considerations that may be used to chose the panel include, but are not limited to the known presence of a compound in or produced by the algae similar to the chosen precursor compound, and the type of metabolism, such as heterotrophic, autotrophic or mixotropic.

The particular non-prokaryotic microalgae employed in the initial panel is not critical to the present invention. Examples of microalgae that can be employed in the present invention include Charophyta, such as Zygenematophyceae (which includes Actinotaenium, Arthrodesmus, Bambuisina, Closterium, Cosmarium, Cosmocladium, Desmidium, Euastrum, Genicularia, Gonatozygon, Heimansia, Hyalotheca, Mesotaenium, Micrasterias, Mougeotia; Netrium, Onychonema, Penium, Phymatodocis, Pleurotaenium, Roya, Sphaerozosma, Spirogyra, Spondylosium, Staurastrum, Staurodesmus, Teilingia, Triploceras, Xanthidium, Zygnema, Zygogonium, Chlorokybophyceae, (which includes Chlorokybus)), Mesostigmatophyceae (which includes Chaetosphaeridium and Mesostigma), Coleochaetophyceae (which includes Coleochaete) and Klebsormidiophyceae (which includes Klebsormidium); Chlorophyta, such as Chlorophyceae (which includes Acetabularia, Acicularia, Actinochloris, Amphikrikos, Anadyomene, Ankistrodesmus, Ankyra, Aphanochaete, Ascochloris, Asterococcus, Asteromonas gracilis, Astrephomene, Atractomorpha, Axilococcus, Axilosphaera, Basichlamys, Basicladia, Binuclearia, Bipedinomonas, Blastophysa, Boergesenia, Boodlea, Borodinella, Borodinellopsis, Botryococcus, Brachiomonas, Bracteacoccus, Bulbochaete, Caespitella, Capsosiphon, Carteria, Centrosphaera, Chaetomorpha, Chaetonema, Chaetopeltis, Chaetophora, Chalmasia, Chamaetrichon, Characiochloris, Characiosiphon, Characium, Chlamydella, Chlamydobotrys, Chlamydocapsa, Chlamydomonas, Chlamydopodium, Chloranomala, Chlorochydridion, Chlorochytrium, Chlorocladus, Chlorocloster, Chlorococcopsis, Chlorococcum, Chlorogonium, Chloromonas, Chlorophysalis, Chlorosarcina, Chlorosarcinopsis, Chlorosphaera, Chlorosphaeropsis, Chlorotetraedron, Chlorothecium, Chodatella, Choricystis, Cladophora, Cladophoropsis, Cloniophora, Closteriopsis, Coccobotrys, Coelastrella, Coelastropsis, Coelastrum, Coenochloris, Coleochlamys, Coronastrum, Crucigenia, Crucigeniella, Ctenocladus, Cylindrocapsa, Cylindrocapsopsis, Cylindrocystis, Cymopolia, Cystococcus, Cystomonas, Dactylococcus, Dasycladus, Deasonia, Derhesia, Desmatractum, Desmodesmus, Desmotetra, Diacanthos, Dicellula, Dicloster, Dicranochaete, Dictyochloris, Dictyococcus, Dictyosphaeria, Dictyosphaerium, Didymocystis, Didymogenes, Dilabifilum, Dimorphococcus, Diplosphaera, Draparnaldia, Dunaliella, Dysmorphococcus, Echinocoleum, Elakatothrix, Enallax, Entocladia, Entransia, Eremosphaera, Ettlia, Eudorina, Fasciculochloris, Fernandinella, Follicularia, Fottea, Franceia, Friedmannia, Fritschiella, Fusola, Geminella, Gloeococcus, Gloeocystis, Gloeodendron, Gloeomonas, Gloeotila, Golenkinia, Gongrosira, Gonium, Graesiella, Granulocystis, Oocystis, Granulocystopsis, Gyorffiana, Haematococcus, Hazenia, Helicodictyon, Hemichloris, Heterochlamydomonas, Heteromastix, Heterotetracystis, Hormidiospora, Hormidium, Hormotila, Hormotilopsis, Hyalococcus, Hyalodiscus, Hyalogonium, Hyaloraphidium, Hydrodictyon, Hypnomonas, Ignatius, Interfilum, Kentrosphaera, Keratococcus, Kermatia, Kirchneriella, Koliella, Lagerheimia, Lautosphaeria, Leptosiropsis, Lobocystis, Lobomonas, Lola, Macrochloris, Marvania, Micractinium, Microdictyon, Microspora, Monoraphidium, Muriella, Mychonastes, Nanochlorum, Nautococcus, Neglectella, Neochloris, Neodesmus, Neomeris, Neospongiococcum, Nephrochlamys, Nephrocytium, Nephrodiella, Oedocladium, Oedogonium, Oocystella, Oonephris, Ourococcus, Pachycladella, Palmella, Palmellococcus, Palmellopsis, Palmodictyon, Pandorina, Paradoxia, Parietochloris, Pascherina, Pauilschulzia, Pectodictyon, Pediastrum, Pedinomotias, Pedinopera, Percursaria, Phacotus, Phaeophila, Physocytium, Pilina, Planctonenma, Planktosphaeria, Platydorina, Platymonas, Pleodorina, Pleurastrum, Pleurococcus, Ploeotila, Polyedriopsis, Polyphysa, Polytoma, Polytomella, Prasinocladus, Prasiococcus, Protoderma, Protosiphon, Pseudendocloniopsis, Pseudocharacium, Pseudochlorella, Pseudochlorococcum, Pseudococconyxa, Pseudodictyosphaerium, Pseudodidymocystis, Pseudokirchneriella, Pseudopleurococcus, Pseudoschizomeris, Pseudoschroederia, Pseudostichococcus, Pseudotetracystis, Pseudotetraëdron, Pseudotrebouxia, Pteromonas, Pulchrasphaera, Pyramimonas, Pyrobotrys, Quadrigula, Radiofilum, Radiosphaera, Raphidocelis, Raphidonenia, Raphidonemopsis, Rhizoclonium, Rhopalosolen, Saprochaete, Scenedesnius, Schizochlamys, Schizomeris, Schroederia, Schroederiella, Scotiellopsis, Siderocystopsis, Siphonocladus, Sirogonium, Sorastrum, Spermatozopsis, Sphaerella, Sphaerellocystis, Sphaerellopsis, Sphaerocystis, Sphaeroplea, Spirotaenia, Spongiochloris, Spongiococcum, Spongiococcuni, Stephanoptera, Stephanosphaera, Stigeoclonium, Struvea, Tetmemorus, Tetrabaena, Tetracystis, Tetradesmus, Tetraedron, Tetrallantos, Tetraselnis, Tetraspora, Tetrastrum, Treubaria, Triploceros, Trochiscia, Trochisciopsis, Ulva, Uronema, Valonia, Valoniopsis, Ventricaria, Viridiella, Vitreochlamys, Volvox, Volvulina, Westella, Willea, Wislouchiella, Zoochlorella, Zygnemopsis, Spermatozopsis, Hyalotheca, Pleurastrum, Chlorococcuni, Chlorella, Pseudopleurococcum, Coelastrum and Rhopalocystis), Ulvophyceae (which includes Acrochaete, Bryopsis, Cephaleuros, Chlorocystis, Enteromorpha, Gloeotilopsis, Halochlorococcum, Ostreobium, Pirula, Pithophora, Planophila, Pseudendoclonium, Trentepohlia, Trichosarcina, Ulothrix, Bolbocoleon, Chaetosiphon, Eugomontia, Oltniannsiellopsis, Pringsheimiella, Pseudodendroclonium, Pseuduilvella, Sporocladopsis, Urospora and Wittrockiella), Trebouxiophyceae (which includes Apatococcus, Asterochloris, Auxetlochlorella, Chlorella, Coccomyxa, Desmococcus, Dictyochloropsis, Elliptochloris, Jaagiella, Leptosira, Lobococcus, Makinoella, Microthamnion, Myrmecia, Nannochloris, Oocystis, Prasiola, Prasiolopsis, Prototheca, Stichococcus, Tetrachlorella, Trebouxia, Trichophilus, Watanabea and Myrmecia), Prasiniophyceae (which includes Bathycoccus, Mantonielia, Micromonas, Nephroselmis, Pseudoscourfieldia, Scherifelia, Picocystis, Pterosperma and Pycnococcus) and Charophyceans (which includes Zygogonium); Diatoms, such as Bolidophyceae (which includes Bolidomonas, Chrysophyceae, Giraudyopsis, Glossomastix, Chromophyton, Chrysamoeba, Chrysochaete, Chrysodidymus, Chrysolepidomonas, Chrysosaccus, Chrysosphaera, Chrysoxys, Cyclonexis, Dinobryon, Epichrysis, Epipyxis, Hibberdia, Lagynion, Lepochromulina, Monas, Monochrysis, Paraphysomonas, Phaeoplaca, Phaeoschizochlamys, Picophagus, Pleurochrysis, Stichogloea and Uroglena), Coscinodiscophyceae (which includes Bacteriastrum, Bellerochea, Biddulphia, Brockmanniella, Corethron, Coscinodiscus, Eucampia, Extubocellulus, Guinardia, Helicotheca, Leptocylindrus, Leyanella, Lithodesmium, Melosira, Minidiscus, Odontella, Planktoniella, Porosira, Proboscia, Rhizosolenia, Stellarima, Thalassionema, Bicosoecid, Symbiomonas, Actinocyclus, Amphora, Arcocellulus, Detonula, Diatoma, Ditylum, Fragilariophyceae, Asterionellopsis, Delphineis, Grammatophora, Nanofrustulum, Synedra and Tabularia), Dinophyceae (which includes Adenoides, Alexandrium, Amphidinium, Ceratium, Ceratocorys, Coolia, Crypthecodinium, Exuviaella, Gambierdiscus, Goniyaulax, Gymnodinium, Gyrodinium, Heterocapsa, Katodinium, Lingulodinium, Pfiesteria, Polarella, Protoceratium, Pyrocystis, Scrippsiella, Symbiodinium, Thecadinium, Thoracosphaera and Zooxanthella) and Alveolates (which includes Cystodinium, Glenodinium, Oxyrrhis, Peridinium, Prorocentrum and Woloszynskia); Rhodophyta, such as Rhodophyceae (which includes Acrochaetium, Agardhiella, Antithamnion, Antithamnionella, Asterocytis, Audouinella, Balbiania, Bangia, Batrachospermum, Bonnemaisonia, Bostrychia, Callithamnion, Caloglossa, Ceramium, Champia, Chroodactylon, Chroothece, Compsopogon, Compsopogonopsis, Cumagloia, Cyanidium, Cystoclonium, Dasya, Digenia, Dixoniella, Erythrocladia, Erythrolobas, Erythrotrichia, Flintiella, Galdieria, Gelidium, Glaucosphaera, Goniotrichum, Gracilaria, Grateloupia, Griffithsia, Hildenbrandia, Hymenocladiopsis, Hypnea, Laingia, Membranoptera, Myriogramme, Nenalion, Nemalionopsis, Neoagardhiella, Palmaria, Phyllophora, Polyneura, Polysiphonia, Porphyra, Porphyridium, Pseudochantransia, Pterocladia, Pugetia, Rhodella, Rhodochaete, Rhodochorton, Rhodosorus, Rhodospora, Rhodymrenia, Seirospora, Selenastrum, Porphyra, Sirodotia, Solieria, Spermothamnion, Spyridia, Stylonema, Thorea, Trailiella and Tuomeya); Cryptophyta, such as Cryptophyceae (which includes Campylomonas, Chilomonas, Chroomonas, Cryptochrysis, Cryptomonas, Goniomonas, Guillardia, Hanusia, Hemiselmis, Plagioselmis, Proteomonas, Pyrenomonas, Rhodomonas and Stroreatula); Chlorarachniophyta, such as Chlorarachnion, Lotharella and Chattonella; Haptophyta, such as Pavlovophyceae (which includes Apistonema, Chrysochromulina, Coccolithophora, Corconitochrysis, Cricosphaera, Diacronema, Emiliana, Pavlova and Ruttnera) and Prymnesiophyceae (which includes Cruciplacolithus, Prymnnesium, Isochrysis, Calyptrosphaera, Chrysotila, Coccolithus, Dicrateria, Heterosigma, Hymenomonas, Imantonia, Gephyrocapsa, Ochrosphaera, Phaeocystis, Platychrysis, Pseudoisochrysis, Syracosphaera and Pleurochrysis); Euglenophyta, such as Euglenophyceae (which includes Astasia, Colacium, Cyclidiopsis, Distigma, Euglena, Eutreptia, Eutreptiella, Gyropaigne, Hyalophacus, Khawkinea Astasia, Lepocinclis, Menoidium, Parmidium, Phacus, Rhabdomonas, Rhabdospira, Tetruetreptia and Trachelomonas); and Heterokonta, such as Phaeophyceae (which includes Ascoseira, Asterocladon, Bodanella, Desmarestia, Dictyocha, Dictyota, Ectocarpus, Halopteris, Heribaudiella, Pleurocladia, Porterinema, Pylaiella, Sorocarpus, Spermatochnus, Sphacelaria and Waerniella), Pelagophyceae (which includes Aureococcus, Aureoumbra, Pelagococcus, Pelagomonas, Pulviniaria and Sarcinochrysis), Xanthophyceae (which includes Asterosiphon, Botrydiopsis, Botrydium, Bumilleria, Bumilleriopsis, Characiopsis, Chlorellidium, Chlorobotrys, Goniochloris, Heterococcus, Heterothrix, Heterotrichella, Mischococcus, Ophiocytium, Pleurochloridella, Plenrochloris, Pseudobumilleriopsis, Sphaerosorus, Tribonema, Vaucheria and Xanthonema), Eustigmatophyceae (which includes Chloridella, Ellipsoidion, Eustigmatos, Monodopsis, Monodus, Nannochloropsis, Polyedriella, Pseudocharaciopsis, Pseudostaurastrum and Vischeria), Syanurophyceae (which includes Mallomonas, Synura and Tessellaria), Phaeothamniophyceae (which includes Phaeobotrys and Phaeothamnion) and Raphidophyceae, (which includes Olisthodiscus, Vacuolaria and Fibrocapsa).

The panel can also include representatives from classes Trebouxiophyceae, Chlorophyceae, Cryptomonideae, Euglenophyta, Raphidophyceae, Diatomatideae and Prasinophyceae.

Partially or fully purified enzymes or cell extracts obtained from the above microalgae, which can be prepared by conventional methods well-known in the art, can also be contacted with the precursor compounds to obtain the desired metabolite (Hellebust and Craigie, eds., “Handbook of Phycological Methods: Phsiological and Biochemical Methods”, University Press, New York, 1978).

B. Precursor Compounds

Precursor compounds may be any organic molecule, including racemic mixtures. The particular compound employed in the present invention as the precursor compound is not critical.

In a preferred embodiment, the precursor compound is a substituted or unsubstituted heterocyclic compound whose heterocyclic ring preferably comprises a 3 to 10 membered heterocyclic ring, more preferably comprises a 4 to 8 membered heterocyclic ring, and most preferably comprises a 5 to 6 membered heterocyclic ring. The heterocyclic ring preferably has a nitrogen, oxygen or sulfur atom as a heteroatom. The heterocyclic ring can be condensed with an aliphatic ring, an aromatic ring or another heterocyclic ring. More preferably, the heterocyclic ring contains 2-7 carbon atoms and 1-3 heteroatoms each selected from the group consisting of oxygen, nitrogen and sulfur. There may be one or more substituent groups on the heterocyclic molecule. The nature of the substituent groups is not critical to the invention. In a preferred embodiment of the invention, the heterocyclic compound has 1-4 substituent groups, each independently selected from the group consisting of hydrogen, hydroxyl, halogen, optionally substituted amino, optionally substituted nitro, optionally substituted sulfo, optionally substituted phospho, optionally substituted alkyl (preferably C₁₋₂₀), optionally substituted cycloaliphatic (preferably C₁₋₂₀), optionally substituted aromatic (preferably C₅₋₂₀), and optionally substituted heterocyclic (preferably C₃₋₂₀) groups.

As the halogen atom substituent, there may be mentioned chloride, bromide, iodide, or fluoride.

As the substituents for the optionally substituted amino group, there may be mentioned, for instance, an optionally substituted C₁₋₂₀ alkyl group, an optionally substituted C₇₋₂₀ aralkyl group, an optionally substituted C₁₋₂₀ acyl group, an optionally substituted C₁₋₂₀ acyl group having an aromatic ring, an optionally substituted acyl group having a heterocyclic group, as exemplified above, and a substituted carbonyl group. As such substituted carbonyl group, there may be mentioned, for instance, an optionally substituted acyl group and a carboxyl group. Typical examples of the optionally substituted amino group include a unsubstituted amino group, an amino group which is substituted with an optionally substituted C₁₋₂₀ alkyl group (for example, methylamino, ethylamino, propylamino, t-butylamino, dimethylamino, diethylamino, dipropylamino, dibutylamino, etc.), an amino group substituted with an optionally substituted C₇₋₂₀ aralkyl group (for instance, benzylamino group and the like), an amino group which is substituted with an optionally substituted C₁₋₂₀ acyl group (for instance, formylamino, acetylamino, valerylamino, isovalerylamino, pivaloylamino, etc.,), an amino group which is substituted with an optionally substituted C₁₋₂₀ acyl group having an aromatic ring (e.g. benzoylamino group, etc.,), an amino group substituted with an optionally substituted acyl group having a heterocyclic ring (for instance, nicotinoylamino group and the like), an amino group which is substituted with a substituted carboxyl group (for instance, acetylamino-methylcarbonylamino, acetylaminoethylcarbonylamino, hydroxymethylcarbonylamino, hydroxyethylcarbonylamino, methoxycarbonylamino, ethoxycarbonylamino group and the like).

As examples of the optionally substituted nitro group, there may be mentioned unsubstituted nitro, nitroso, nitrosooxy, and isothiocyanato groups. As the substituent(s) for the nitro group there may be mentioned for instance, an optionally substituted C₁₋₂₀ alkyl group, an optionally substituted C₇₋₂₀ aralkyl group, an optionally substituted C₁₋₂₀ acyl group, an optionally substituted C₁₋₂₀ acyl group having an aromatic ring, an optionally substituted acyl group having a heterocyclic group, as exemplified above and a substituted carbonyl group. As such substituted carbonyl group, there may be mentioned, for instance, an optionally substituted acyl group and a carboxyl group. Preferred examples include ethyl(hydroxy)oxoammonium, 1-(3-carboxyphenyl)triaza-1,2-dien-2-ium, 3-furyl-N-nitrosomethanaminium, and [(2E)-but-2-enyloxy](hydroxy)oxoammonium.

As examples of the optionally substituted sulfo group there may be mentioned unsubstituted sulfo, sulfino, sulfamoyl, sulfato, and sulfoamino groups. Examples of the sulfo group substituent include, for instance, an aralkylsulfonyl group such as a C₁₋₂₀ alkylsulfonyl group which may be substituted with, for instance, a C₁₋₂₀ alkoxy group, a C₁₋₂₀ alkoxy-C₁₋₂₀ alkoxy group, a C₇₋₂₀ aralkyloxy group, a benzoyl group, a C₁₋₄ alkylthio group and a halogen atom (e.g. methanesulfonyl, ethanesulfonyl, propanesulfonyl, butanesulfonyl, trichloro methanesulfonyl, trifluoromethanesulfonyl, etc.); an optionally substituted arylsulfonyl group including a C₆₋₂₀ arylsulfonyl group which may be substituted with, for example, a C₁₋₂₀ alkyl group, a hydroxyl group, a C₁₋₂₀ alkoxy group, a nitro group or a halogen atom, such as benzenesulfonyl, m-nitrobenzenesulfonyl, p-nitrobenzenesulfonyl, p-chlorobenzenesulfonyl, p-brnmobenzenesulfonyl, p-toluenesulfonyl, naphthalene-sulfonyl and etc.

As examples of the optionally substituted phospho group, there may be mentioned unsubstituted phosphato, phosphito, diethylphosphono, and pentafluorophosphato groups. The optional substituents for the phospho group include, for instance, an optionally substituted C₁₋₂₀ alkyl group, an optionally substituted C₇₋₂₀ aralkyl group, an optionally substituted C₁₋₂₀ acyl group, an optionally substituted C₁₋₂₀ acyl group having an aromatic ring, an optionally substituted acyl group having a heterocyclic group, as exemplified above, and a substituted carbonyl group. As such substituted carbonyl group, there may be mentioned, for instance, an optionally substituted acyl group and a carboxyl group. Preferrable examples include hydroxy(1-methylbutyl)oxophosphonium, hydroxy(1H-inden-1-ylmethyl)oxophosphonium, {[2-(chloromethyl)-2-methylbut-3-enyl]oxy}(hydroxy)oxophosphonium, or adenosine phosphatidyl groups.

As the optionally substituted alkyl group having 1 to 20 carbon atoms there may be mentioned methyl, ethyl, propyl, isopropyl, butyl, isobutyl, s-butyl and t-butyl groups. Examples of the substituent(s) for the C₁₋₂₀ alkyl group include a hydroxyl group, a C₁₋₂₀ alkoxy group, a benzoyl group, a C₂₋₂₀ allyl group (e.g. a butadienyl group) a C₆₋₁₂ aryl group (e.g. phenyl is group) which may be substituted with a substituent (for example, a C₁₋₂₀ alkoxy group, etc.), a C₁₋₂₀ alkylthio group and a halogen atom. As examples of such substituted C₁₋₂₀ alkyl groups, there may be mentioned a C₁₋₂₀ alkyl group substituted with hydroxyl group(s) (for example, hydroxymethyl, 2-hydroxyethyl, 1,2-dihydroxyethyl, 2,2-dihydroxyethyl, 3,3-dihydroxypropyl group, etc.), a C₁₋₂₀ alkoxy-C₁₋₂₀ alkyl group (for instance, methoxymethyl, ethoxymethyl, t-butoxymethyl, 1-ethoxyethyl, 2-methoxyethyl group, etc.), phenacyl group, a C₁₋₂₀ alkylthio-C₁₋₂₀ alkyl group (e.g. a C₁₋₂₀ alkylthiomethyl such as methylthiomethyl, ethylthiomethyl group, etc.), a C₁₋₂₀ haloalkyl group having 1 or more of halogen atoms such as chloromethyl, 2-chloroethyl, 3-chloropropyl, 4-chlorobutyl, dichloromethyl, trichloromethyl, trifluoromethyl, 2,2,2-trichloroethyl, 2,2,2-trifluoroethyl, 1,1,2,2,2-pentafluoroethyl, and etc.

As optionally substituted cycloaliphatic groups there may be mentioned cyclopropyl, cyclobutyl, cyclopentyl and cyclohexyl. Examples of the substituents for the optionally substituted cycloaliphatic group include an optionally substituted alkyl group, an optionally substituted allyl group, an optionally substituted cycloalkyl group, an optionally substituted heterocyclic group, and an optionally substituted aralkyl group.

The optionally substituted alkyl group includes, for example, an optionally substituted alkyl group having 1 to 20 carbon atoms such as methyl, ethyl, n-propyl, i-propyl, n-butyl, i-butyl, s-butyl and t-butyl groups. The substituents for the C₁₋₂₀ alkyl group include, for example, a C₁₋₂₀ alkoxy group, a C₁₋₂₀ alkoxy-C₁₋₂₀ alkoxy group, and a C₇₋₂₀ aralkyloxy group.

Substituents for the allyl group include, for instance, substituents for the C₁₋₂₀ alkyl group mentioned above.

Examples of the optionally substituted cycloalkyl group include a cycloalkyl group having 3 to 10 carbon atoms such as cyclopropyl, cyclopentyl, cyclohexyl, cyclobeptyl, cyclooctyl, cyclononyl and cyclo-decyl groups. The substituent(s) for the cycloalkyl group include, for example, a halogen atom, a C₁₋₂₀ alkyl group, and a hydroxyl group.

As the optionally substituted heterocyclic group, there may be mentioned, for example, an optionally substituted 3 to 10-membered heterocyclic group having, other than carbon atoms, 1 to 3 atoms of oxygen, sulfur or nitrogen as hetero atom(s). The optionally substituted heterocyclic group may frequently be a non-aromatic perhydroheterocyclic group. The 5- or 6-membered heterocyclic group includes, for instance, tetrahydrofuranyl, tetrahydrothiofuranyl, tetrahydropyranyl and tetrahydrothiopyranyl groups. Examples of the substituents for the heterocyclic group include a halogen atom, a C₁₋₂₀ alkyl group, a C₁₋₂₀ alkoxy group such as methoxy, ethoxy, propoxy, isopropoxy, butoxy, isobutoxy, s-butoxy and t-butoxy, and substituents as mentioned above for the alkyl group.

Preferable examples of the optionally substituted heterocyclic group include an optionally substituted tetrahydropyranyl group (e.g. tetrahydropyranyl, 3-bromotetrahydropyranyl, 4-methnxytetrahydropyranyl, etc.), an optionally substituted tetrahydrothiopyranyl group (for example, tetrahydrothiopyranyl, 3-bromotetrahydrothiopyranyl, 4-ethoxytetrahydrothiopyranyl, etc.), an optionally substituted tetrahydrofuranyl group (for instance, tetrahydrofuranyl, etc.), and an optionally substituted tetrahydrothiofuranyl group (e.g., tetrahydrothiofuranyl).

Examples of the optionally substituted aralkyl group include an optionally substituted aralkyl group having 7 to 20 carbon atoms (e.g., benzyl, etc.). The substituent for the aralkyl group includes, for instance, a C₁₋₂₀ alkyl group; a C₆₋₁₂ aryl group such as phenyl group; a hydroxyl group, a C₁₋₂₀ alkoxy group; a nitro group; and a halogen atom.

Examples of the optionally substituted aralkyl group include benzyl, o-chlorobenzyl, o-nitrobenzyl, p-chlorobenzyl, p-methoxybenzyl, p-methylbenzyl, p-nitrobenzyl, 2,6-dichlorobenzyl, diphenylmethyl, trityl and the like.

Heterocyclic compounds comprise a class of commercially important molecules. Many drugs, pigments, vitamins, and additional nutraceuticals contain heterocyclic rings in their structures (Wackett et al, supra; see also Delgado et al, Wilson and Gisvold's Textbook of Organic, Medicinal, and Pharmaceutical Chemistry, Lippincott-Raven, Philadelphia, Pa. (1998)). Examples of commercially valuable heterocyclic compounds include the vitamin biotin, the anti-neoplastic agent 8-azaguanine, the antibiotic penicillin, and the industrial solvent tetrahydrofuran. Improved methods for generating and opening heterocyclic rings are valuable in their synthesis, as well as in their ecologically safe disposal.

Particularly preferred heterocyclic compounds are the oxazolidines, represented by

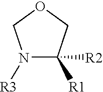

wherein R¹, R² and R³ are each independently selected from the group consisting of hydrogen, hydroxyl, halogen, optionally substituted amino, optionally substituted nitro, optionally substituted sulfo, optionally substituted phospho, optionally substituted alkyl (C₁₋₂₀), optionally substituted cycloaliphatic (C₁₋₂₀), optionally substituted aromatic (C₅₋₂₀), and optionally substituted heterocyclic (C₃₋₂₀) groups.

As the halogen atom substituent, there may be mentioned chloride, bromide, iodide, or fluoride.

As the substituents for the optionally substituted amino group, there may be mentioned, for instance, an optionally substituted C₁₋₂₀ alkyl group, an optionally substituted C₇₋₂₀ aralkyl group, an optionally substituted C₁₋₂₀ acyl group, an optionally substituted C₁₋₂₀ acyl group having an aromatic ring, an optionally substituted acyl group having a heterocyclic group, as exemplified above and a substituted carbonyl group. As such substituted carbonyl group, there may be mentioned, for instance, an optionally substituted acyl group and a carboxyl group. Typical examples of the optionally substituted amino group include an amino group, an amino group which is substituted with an optionally substituted C₁₋₂₀ alkyl group (for example, methylamino, ethylamino, propylamino, t-butylamino, dimethylamino, diethylamino, dipropylamino, dibutylamino, etc.), an amino group substituted with an optionally substituted C₇₋₂₀ aralkyl group (for instance, benzylamino group and the like), an amino group which is substituted with an optionally substituted C₁₋₂₀ acyl group (for instance, formylamino, acetylamino, valerylamino, isovalerylamino, pivaloylamino, etc.,), an amino group which is substituted with an optionally substituted C₁₋₂₀ acyl group having an aromatic ring (e.g., benzoylamino group, etc.,), an amino group substituted with an optionally substituted acyl group having a heterocyclic ring (for instance, nicotinoylamino group and the like), an amino group which is substituted with a substituted carboxyl group (for instance, acetylamino-methylcarbonylamino, acetylaminoethylcarbonylamino, hydroxymethylcarbonylamino, hydroxyethylcarbonylamino, methoxycarbonylamino, ethoxycarbonylamino group and the like).

As examples of the optionally substituted nitro group, there may be mentioned unsubstituted nitro, nitroso, nitrosooxy, and isothiocyanato groups. As the substituent(s) for the nitro group there may be mentioned for instance, an optionally substituted C₁₋₂₀ alkyl group, an optionally substituted C₇₋₂₀ aralkyl group, an optionally substituted C₁₋₂₀ acyl group, an optionally substituted C₁₋₂₀ acyl group having an aromatic ring, an optionally substituted acyl group having a heterocyclic group, as exemplified above and a substituted carbonyl group. As such substituted carbonyl group, there may be mentioned, for instance, an optionally substituted acyl group and a carboxyl group. Preferred examples include ethyl(hydroxy)oxoammonium, 1-(3-carboxyphenyl)triaza-1,2-dien-2-ium, 3-furyl-N-nitrosomethanaminium, and [(2E)-but-2-enyloxy](hydroxy)oxoammonium.

As examples of the optionally substituted sulfo group there may be mentioned unsubstituted sulfo, sulfino, sulfamoyl, sulfato, and sulfoamino groups. Examples of the sulfo group substituent include, for instance, an aralkylsulfonyl group such as a C₁₋₂₀ alkylsulfonyl group which may be substituted with, for instance, a C₁₋₂₀ alkoxy group, a C₁₋₂₀ alkoxy-C₁₋₂₀ alkoxy group, a C₇₋₂₀ aralkyloxy group, a benzoyl group, a C₁₋₄ alkylthio group and a halogen atom (e.g. methanesulfonyl, ethanesulfonyl, propanesulfonyl, butanesulfonyl, trichloro methanesulfonyl, trifluoromethanesulfonyl, etc.); an optionally substituted arylsulfonyl group including a C₆₋₂₀ arylsulfonyl group which may be substituted with, for example, a C₁₋₂₀ alkyl group, a hydroxyl group, a C₁₋₂₀ alkoxy group, a nitro group or a halogen atom, such as benzenesulfonyl, m-nitrobenzenesulfonyl, p-nitrobenzenesulfonyl, p-chlorobenzenesulfonyl, p-bromobenzenesulfonyl, p-toluenesulfonyl, naphthalene-sulfonyl and etc.

As examples of the optionally substituted phospho group, there may be mentioned unsubstituted phospho, phosphato, phosphito, diethylphosphono, and pentafluorophosphato groups. The optional substituents for the phospho group include, for instance, an optionally substituted C₁₋₂₀ alkyl group, an optionally substituted C₇₋₂₀ aralkyl group, an optionally substituted C₁₋₂₀ acyl group, an optionally substituted C₁₋₂₀ acyl group having an aromatic ring, an optionally substituted acyl group having a heterocyclic group, as exemplified above, and a substituted carbonyl group. As such substituted carbonyl group, there may be mentioned, for instance, an optionally substituted acyl group and a carboxyl group. Preferrable examples include hydroxy(1-methylbutyl)oxophosphonium, hydroxy(1H-inden-1-ylmethyl)oxophosphonium, {[2-(chloromethyl)-2-methylbut-3-enyl]oxy}(hydroxy)oxophosphonium, or adenosine phosphatidyl groups.

As the optionally substituted alkyl group having 1 to 20 carbon atoms there may be mentioned methyl, ethyl, propyl, isopropyl, butyl, isobutyl, s-butyl and t-butyl groups. Examples of the substituent(s) for the C₁₋₂₀ alkyl group include a hydroxyl group, a C₁₋₂₀ alkoxy group, a benzoyl group, a C₂₋₂₀ allyl group (e.g. a butadienyl group) a C₆₋₁₂ aryl group (e.g. phenyl group) which may be substituted with a substituent (for example, a C₁₋₂₀ alkoxy group, etc.), a C₁₋₂₀ alkylthio group and a halogen atom. As examples of such substituted C₁₋₂₀ alkyl groups, there may be mentioned a C₁₋₂₀ alkyl group substituted with hydroxyl group(s) (for example, hydroxymethyl, 2-hydroxyethyl, 1,2-dihydroxyethyl, 2,2-dihydroxyethyl, 3,3-dihydroxypropyl group, etc.), a C₁₋₂₀ alkoxy-C₁₋₂₀ alkyl group (for instance, methoxymethyl, ethoxymethyl, t-butoxymethyl, 1-ethoxyethyl, 2-methoxyethyl group, etc.), phenacyl group, a C₁₋₂₀ alkylthio-C₁₋₂₀ alkyl group (e.g. a C₁₋₂₀ alkylthiomethyl such as methylthiomethyl, ethylthiomethyl group, etc.), a C₁₋₂₀ haloalkyl group having 1 or more of halogen atoms such as chloromethyl, 2-chloroethyl, 3-chloropropyl, 4-chlorobutyl, dichloromethyl, trichloromethyl, trifluoromethyl, 2,2,2-trichloroethyl, 2,2,2-trifluoroethyl, 1,1,2,2,2-pentafluoroethyl, and etc.

As optionally substituted cycloaliphatic groups there may be mentioned cyclopropyl, cyclobutyl, cyclopentyl and cyclohexyl. Examples of the substituents for the optionally substituted cycloaliphatic group include an optionally substituted alkyl group, an optionally substituted allyl group, an optionally substituted cycloalkyl group, an optionally substituted heterocyclic group, and an optionally substituted aralkyl group.

The optionally substituted alkyl group includes, for example, an optionally substituted alkyl group having 1 to 20 carbon atoms such as methyl, ethyl, n-propyl, i-propyl, n-butyl, i-butyl, s-butyl and t-butyl groups. The substituents for the C₁₋₂₀ alkyl group include, for example, a C₁₋₂₀ alkoxy group, a C₁₋₂₀ alkoxy-C₁₋₂₀ alkoxy group and a C₇₋₂₀ aralkyloxy group.

Substituents for the allyl group include, for instance, substituents for the C₁₋₂₀ alkyl group mentioned above.

Examples of the optionally substituted cycloalkyl group include a cycloalkyl group having 3 to 10 carbon atoms such as cyclopropyl, cyclopentyl, cyclohexyl, cyclobeptyl, cyclooctyl, cyclononyl and cyclodecyl groups. The substituent(s) for the cycloalkyl group include, for example, a halogen atom, a C₁₋₂₀ alkyl group, and a hydroxyl group.

As the optionally substituted heterocyclic group, there may be mentioned, for example, an optionally substituted 3 to 10-membered heterocyclic group having, other than carbon atoms, 1 to 3 atoms of oxygen, sulfur or nitrogen as hetero atom(s). The optionally substituted heterocyclic group may be a non-aromatic perhydroheterocyclic group. The 5- or 6-membered heterocyclic group includes, for instance, tetrahydrofuranyl, tetrahydrothiofuranyl, tetrahydropyranyl and tetrahydrothiopyranyl groups. Examples of the substituents for the heterocyclic group include a halogen atom, a C₁₋₂₀ alkyl group, a C₁₋₂₀ alkoxy group such as methoxy, ethoxy, propoxy, isopropoxy, butoxy, isobutoxy, s-butoxy and t-butoxy, and substituents as mentioned above for the alkyl group.

Preferable examples of the optionally substituted heterocyclic group include an optionally substituted tetrahydropyranyl group (e.g., tetrahydropyranyl, 3-bromotetrahydropyranyl, 4-methnxytetrahydropyranyl, etc.), an optionally substituted tetrahydrothiopyranyl group (for example, tetrahydrothiopyranyl, 3-bromotetrahydrothiopyranyl, 4-methoxytetrahydrothiopyranyl, etc.), an optionally substituted tetrahydrofuranyl group (for instance, tetrahydrofuranyl, etc.), and an optionally substituted tetrahydrothiofuranyl group (e.g. tetrahydrothiofuranyl).

Examples of the optionally substituted aralkyl group include an optionally substituted aralkyl group having 7 to 20 carbon atoms (e.g., benzyl, etc.). The substituent for the aralkyl group includes, for instance, a C₁₋₂₀ alkyl group; a C₆₋₁₂ aryl group such as phenyl group; a hydroxyl group, a C₁₋₂₀ alkoxy group; a nitro group; and a halogen atom.

Examples of the optionally substituted aralkyl group include benzyl, o-chlorobenzyl, o-nitrobenzyl, p-chlorobenzyl, p-methoxybenzyl, p-methylbenzyl, p-nitrobenzyl, 2,6-dichlorobenzyl, diphenylmethyl, trityl and the like.

Specific non-limiting examples of heterocyclic compounds which can be employed in the present invention as the precursor compound include substituted 5-membered rings containing 2 heteroatoms selected from the group consisting of oxygen, nitrogen and sulfur, preferably, imidazolidines, oxazolidines or thiazolidines, wherein the substituents are as defined above. Additionally, there can be employed as the precursor compound substituted 3-membered rings containing 1 heteroatom selected from the group consisting of oxygen and nitrogen, preferably 2-substituted oxiranyl compounds wherein the substituents are as defined above.

In another preferred embodiment, the precursor compound is a substituted or unsubstituted heterochain compound whose backbone consists of 4 to 12 carbon atoms and 1-3 heteroatoms, preferably nitrogen, oxygen, phosphorus or sulfur (hereinafter referred to as a “heterochain”). The heterochain can be condensed with an aliphatic ring, an aromatic ring or a heterocyclic ring. Most prefereably, the heterochain contains 3-6 carbon atoms and 1-4 heteroatoms each selected from the group consisting of oxygen, nitrogen and sulfur. There may be one or more substituent groups on the precursor molecule. The nature of the substituent groups is not critical to the invention.

The substituent groups are indepently selected from the group consisting of hydrogen, hydroxyl, halogen, optionally substituted amino, optionally substituted nitro, optionally substituted sulfo, optionally substituted phospho, optionally substituted alkyl (preferably C₁₋₂₀), optionally substituted cycloaliphatic (preferably C₁₋₂₀), optionally substituted aromatic (preferably C₅₋₂₀), and optionally substituted heterocyclic (preferably C₃₋₂₀) groups.

As the halogen atom substituent, there may be mentioned chloride, bromide, iodide, or fluoride.

As the substituents for the optionally substituted amino group, there may be mentioned, for instance, an optionally substituted C₁₋₂₀ alkyl group, an optionally substituted C₇₋₂₀ aralkyl group, an optionally substituted C₁₋₂₀ acyl group, an optionally substituted C₁₋₂₀ acyl group having an aromatic ring, an optionally substituted acyl group having a heterocyclic group, as exemplified above, and a substituted carbonyl group. As such substituted carbonyl group, there may be mentioned, for instance, an optionally substituted acyl group and a carboxyl group. Typical examples of the optionally substituted amino group include a unsubstituted amino group, an amino group which is substituted with an optionally substituted C₁₋₂₀ alkyl group (for example, methylamino, ethylamino, propylamino, t-butylamino, dimethylamino, diethylamino, dipropylamino, dibutylamino, etc.), an amino group substituted with an optionally substituted C₇₋₂₀ aralkyl group (for instance, benzylamino group and the like), an amino group which is substituted with an optionally substituted C₁₋₂₀ acyl group (for instance, formylamino, acetylamino, valerylamino, isovalerylamino, pivaloylamino, etc.), an amino group which is substituted with an optionally substituted C₁₋₂₀ acyl group having an aromatic ring (e.g. benzoylamino group, etc.), an amino group substituted with an optionally substituted acyl group having a heterocyclic ring (for instance, nicotinoylamino group and the like), an amino group which is substituted with a substituted carboxyl group (for instance, acetylamino-methylcarbonylamino, acetylaminoethylcarbonylamino, hydroxymethylcarbonylamino, hydroxyethylcarbonylamino, methoxycarbonylamino, ethoxycarbonylamino group and the like).

As examples of the optionally substituted nitro group, there may be mentioned unsubstituted nitro, nitroso, nitrosooxy, and isothiocyanato groups. As the substituent(s) for the nitro group there may be mentioned for instance, an optionally substituted C₁₋₂₀ alkyl group, an optionally substituted C₇₋₂₀ aralkyl group, an optionally substituted C₁₋₂₀ acyl group, an optionally substituted C₁₋₂₀ acyl group having an aromatic ring, an optionally substituted acyl group having a heterocyclic group, as exemplified above and a substituted carbonyl group. As such substituted carbonyl group, there may be mentioned, for instance, an optionally substituted acyl group and a carboxyl group. Preferred examples include ethyl(hydroxy)oxoammonium, 1-(3-carboxyphenyl)triaza-1,2-dien-2-ium, 3-furyl-N-nitrosomethanaminium, and [(2E)-but-2-enyloxy](hydroxy)oxoammonium.

As examples of the optionally substituted sulfo group there may be mentioned unsubstituted sulfo, sulfino, sulfamoyl, sulfato, and sulfoamino groups. Examples of the sulfo group substituent include, for instance, an aralkylsulfonyl group such as a C₁₋₂₀ alkylsulfonyl group which may be substituted with, for instance, a C₁₋₂₀ alkoxy group, a C₁₋₂₀ alkoxy-C₁₋₂₀ alkoxy group, a C₇₋₂₀ aralkyloxy group, a benzoyl group, a C₁₋₄ alkylthio group and a halogen atom (e.g. methanesulfonyl, ethanesulfonyl, propanesulfonyl, butanesulfonyl, trichloro methanesulfonyl, trifluoromethanesulfonyl, etc.); an optionally substituted arylsulfonyl group including a C₆₋₂₀ arylsulfonyl group which may be substituted with, for example, a C₁₋₂₀ alkyl group, a hydroxyl group, a C₁₋₂₀ alkoxy group, a nitro group or a halogen atom, such as benzenesulfonyl, m-nitrobenzenesulfonyl, p-nitrobenzenesulfonyl, p-chlorobenzenesulfonyl, p-bromobenzenesulfonyl, p-toluenesulfonyl, naphthalene-sulfonyl and etc.

As examples of the optionally substituted phospho group, there may be mentioned unsubstituted phosphato, phosphito, diethylphosphono, and pentafluorophosphato groups. The optional substituents for the phospho group include, for instance, an optionally substituted C₁₋₂₀ alkyl group, an optionally substituted C₇₋₂₀ aralkyl group, an optionally substituted C₁₋₂₀ acyl group, an optionally substituted C₁₋₂₀ acyl group having an aromatic ring, an optionally substituted acyl group having a heterocyclic group, as exemplified above, and a substituted carbonyl group. As such substituted carbonyl group, there may be mentioned, for instance, an optionally substituted acyl group and a carboxyl group. Preferrable examples include hydroxy(1-methylbutyl)oxophosphonium, hydroxy(1H-inden-1-ylmethyl)oxophosphonium, {[2-(chloromethyl)-2-methylbut-3-enyl]oxy}(hydroxy)oxophosphonium, or adenosine phosphatidyl groups.

As the optionally substituted alkyl group having 1 to 20 carbon atoms there may be mentioned methyl, ethyl, propyl, isopropyl, butyl, isobutyl, s-butyl and t-butyl groups. Examples of the substituent(s) for the C₁₋₂₀ alkyl group include a hydroxyl group, a C₁₋₂₀ alkoxy group, a benzoyl group, a C₂₋₂₀ allyl group (e.g. a butadienyl group) a C₆₋₁₂ aryl group (e.g. phenyl group) which may be substituted with a substituent (for example, a C₁₋₂₀ alkoxy group, etc.), a C₁₋₂₀ alkylthio group and a halogen atom. As examples of such substituted C₁₋₂₀ alkyl groups, there may be mentioned a C₁₋₂₀ alkyl group substituted with hydroxyl group(s) (for example, hydroxymethyl, 2-hydroxyethyl, 1,2-dihydroxyethyl, 2,2-dihydroxyethyl, 3,3-dihydroxypropyl group, etc.), a C₁₋₂₀ alkoxy-C₁₋₂₀ alkyl group (for instance, methoxymethyl, ethoxymethyl, t-butoxymethyl, 1-ethoxyethyl, 2-methoxyethyl group, etc.), phenacyl group, a C₁₋₂₀ alkylthio-C₁₋₂₀ alkyl group (e.g. a C₁₋₂₀ alkylthiomethyl such as methylthiomethyl, ethylthiomethyl group, etc.), a C₁₋₂₀ haloalkyl group having 1 or more of halogen atoms such as chloromethyl, 2-chloroethyl, 3-chloropropyl, 4-chlorobutyl, dichloromethyl, trichloromethyl, trifluoromethyl, 2,2,2-trichloroethyl, 2,2,2-trifluoroethyl, 1,1,2,2,2-pentafluoroethyl, and etc.

As optionally substituted cycloaliphatic groups there may be mentioned cyclopropyl, cyclobutyl, cyclopentyl and cyclohexyl. Examples of the substituents for the optionally substituted cycloaliphatic group include an optionally substituted alkyl group, an optionally substituted allyl group, an optionally substituted cycloalkyl group, an optionally substituted heterocyclic group, and an optionally substituted aralkyl group.

The optionally substituted alkyl group includes, for example, an optionally substituted alkyl group having 1 to 20 carbon atoms such as methyl, ethyl, n-propyl, i-propyl, n-butyl, i-butyl, s-butyl and t-butyl groups. The substituents for the C₁₋₂₀ alkyl group include, for example, a C₁₋₂₀ alkoxy group, a C₁₋₂₀ alkoxy-C₁₋₂₀ alkoxy group, and a C₇₋₂₀ aralkyloxy group.

Substituents for the allyl group include, for instance, substituents for the C₁₋₂₀ alkyl group mentioned above.

Examples of the optionally substituted cycloalkyl group include a cycloalkyl group having 3 to 10 carbon atoms such as cyclopropyl, cyclopentyl, cyclohexyl, cyclobeptyl, cyclooctyl, cyclononyl and cyclo-decyl groups. The substituent(s) for the cycloalkyl group include, for example, a halogen atom, a C₁₋₂₀ alkyl group, and a hydroxyl group.

As the optionally substituted heterocyclic group, there may be mentioned, for example, an optionally substituted 3 to 10-membered heterocyclic group having, other than carbon atoms, 1 to 3 atoms of oxygen, sulfur or nitrogen as hetero atom(s). The optionally substituted heterocyclic group may frequently be a non-aromatic perhydroheterocyclic group. The 5- or 6-membered heterocyclic group includes, for instance, tetrahydrofuranyl, tetrahydrothiofuranyl, tetrahydropyranyl and tetrahydrothiopyranyl groups. Examples of the substituents for the heterocyclic group include a halogen atom, a C₁₋₂₀ alkyl group, a C₁₋₂₀ alkoxy group such as methoxy, ethoxy, propoxy, isopropoxy, butoxy, isobutoxy, s-butoxy and t-butoxy, and substituents as mentioned above for the alkyl group.

Preferable examples of the optionally substituted heterocyclic group include an optionally substituted tetrahydropyranyl group (e.g., tetrahydropyranyl, 3-bromotetrahydropyranyl, 4-methnxytetrahydropyranyl, etc.), an optionally substituted tetrahydrothiopyranyl group (for example, tetrahydrothiopyranyl, 3-bromotetrahydrothiopyranyl, 4-ethoxytetrahydrothiopyranyl, etc.), an optionally substituted tetrahydrofuranyl group (for instance, tetrahydrofuranyl, etc.), and an optionally substituted tetrahydrothiofuranyl group (e.g. tetrahydrothiofuranyl).

Examples of the optionally substituted aralkyl group include an optionally substituted aralkyl group having 7 to 20 carbon atoms (e.g. benzyl, etc.). The substituent for the aralkyl group includes, for instance, a C₁₋₂₀ alkyl group; a C₆₋₁₂ aryl group such as phenyl group; a hydroxyl group, a C₁₋₂₀ alkoxy group; a nitro group; and a halogen atom.

Examples of the optionally substituted aralkyl group include benzyl, o-chlorobenzyl, o-nitrobenzyl, p-chlorobenzyl, p-methoxybenzyl, p-methyl-benzyl, p-nitrobenzyl, 2,6-dichlorobenzyl, diphenylmethyl, trityl and the like.

Particularly preferred heterochain compounds are N-substituted amides of the general formula

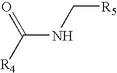

wherein R₄, and R₅ are each independently selected from the group consisting of hydrogen, hydroxyl, halogen, optionally substituted amino, optionally substituted nitro, optionally substituted sulfo, optionally substituted phospho, optionally substituted alkyl (C₁₋₂₀), optionally substituted cycloaliphatic (C₁₋₂₀), optionally substituted aromatic (C₅₋₂₀), and optionally substituted heterocyclic (C₃₋₂₀) groups.

As the aforementioned substituents, there may be mentioned the same groups are described for oxazolidine substituents.

Heterochain compounds comprise a class of commercially important compounds. For example, they include peptides such as the dipeptide Nutrasweet, and the solvent ether. In addition, they are valuable starting compounds for the synthesis of industrial chemicals and pharmaceuticals. They are often used in as the starting point in the synthesis of symmetric inhibitors of the AIDS protease (i.e., Kempf et al, Proc. Natl. Acad. Sci., USA, 92:2484 (1995)). Specific examples include ritonavir, an HIV protease inhibitor; mitoguazone, an anti-timuor drug; bethanechol chloride, an anti-muscarinic receptor; and the hypoglycemic agents galegine and synthalin, which also have anti-trypanosomal properties (Delgado et al, supra).

C. The Solvent

In the method of the present invention, algae are eliminated from the initial panel based on their growth characteristics in media containing a solvent for the precursor compound.

Each algae is grown in the presence and absence of the same amount of solvent for the precursor compound to be used in later experiments. The final concentration of solvent in the culture medium is not critical to the present invention, and preferably is in the range from 0 to 10%, more preferably 0-1.0%. Various solvents and incubation conditions can be tested in order to optimize growth of the microalgae.

The particular solvent employed in not critical to the present invention. Examples of such solvents include dimethylformamide, methanol and benzene. Water and methanol are preferred solvents.

D. Algal Growth Medium

The particular algal growth medium employed is not critical to the present invention. Examples of such growth medium include those obtained from The Culture Collection of Algae and Protozoa, www.ife.ac.uk/ccap/mediarecipes (i.e., 2ASW (double strength Artificial Seawater), 2SNA (Saline Seawater Nutrient Agar), AJS (Acidified JM:SE), ANT (Antia's Medium), ASW (Artificial Seawater), ASW+barley (Artificial Seawater+barley grains), ASW:BG, ASW:SES, ASWP (Artificial Seawater for Protozoa), BB (Bold's Basal Medium), BB:Merds, CH (Chalkley's Medium), CHM (Chilomonas Medium), CMA (Corn Meal Glucose Agar), DM (Diatom Medium), E27 (E27 Medium), E31 (E31 Medium), E31:ANT, EG (Euglena Gracilis Medium), EG:JM, Euglena Medium with Minerals, f/2+Si (f/2 Medium+sodium metasilicate), HSM (Jones's Horse Serum Medium), JM (Jaworski's Medium), JM: SE, K Medium, MC (Modified Chang's Serum-Casein-Glucose-Yeast Extract (1) Medium), MCH (Modified Chalkley's Medium), MErds (Modified Foyns Erdschreiber Medium), MErds/MY75S, MP (Chapman-Andresen's Modified Pringsheim's Solution), MW (Mineral Water), MWC (Modified Woods Hole Medium), MY75S (Malt & Yeast Extract-75% Seawater Agar), NN (Non-Nutrient (Amoeba Saline) Agar), NSW (Natural Seawater), PC (Prescott's and Carrier's Solution), PE (Plymouth Erdschreiber Medium), PER (Peranema Medium), PJ (Prescott's & James's Solution), PJ/NN, PP (Proteose Peptone Medium), PPG (Proteose Peptone Glucose Medium), PPY (Proteose Peptone Yeast Extract Medium), S/W (Soil/Water Biphasic Medium), S/W+AMP (Soil/Water Biphasic Medium+ammonium magnesium phosphate), S/W+Ca (Soil/Water Biphasic Medium+calcium carbonate), S75S (Sigma Cereal Leaf-75% Seawater), S75S:NSW, S77+vitamins (S77 Medium+vitamins), S88+vitamins (S88 Medium+vitamins), SE (Soil Extract), SE1 (Soil Extract 1), SE2 (Soil Extract 2), SES (Soil Extract with Added Salts), SES:MP, SNA (Seawater Nutrient Agar), SNA/5 (Brackish Seawater Nutrient Agar), SPA (Sigma Cereal Leaf-Prescott Agar), Spirulina Medium, SPL (Sigma Cereal Leaf-Prescott Liquid), SPL/0.01% SPA, SPL:MP, SPL:PJ, SPL:PJ/0.01% SPA, UM (Uronema Medium), Walne's (Walne's Medium) and YEL (Yeast Extract-Liver Digest Medium); Culture Maintenance U Toronto, www.botany.utoronto/utcc/.ca (i.e., BBM, CHU-10, ESAW, f/2 VITAMIN and Modified Acid Medium); UTEX: The Culture Collection of Algae, www.bio.utexas.edu/research/utex/media (i.e., 1/2ES-Enriched seawater, 2/3ES-Enriched seawater, 2X-seawater, Allen medium, Artificial seawater, AS100, Bold INV, Bold 3NV, Bristol-NaCl, Bristol's Solution/Medium, Chu, Cyanidium, Cyanophycean, Desmid, DYIII, Erdschrieber, ES/10-Enriched seawater, ES/2-Enriched seawater, ES/4-Enriched seawater, ES-Enriched seawater, Euglena, HEPES-volvox, J medium, LDM, Malt, MES-Volvox, NBB, Ochromonas Medium, P49, Pasteurized seawater, Polytomella, Porphyridium Medium, Proteose, Soil extract, Soilwater, Soilwater (BAR), Soilwater (GR−) supernatant, Soilwater (GR+), Soilwater (GR+) supernatant, Soilwater (PEA), Soilwater (Peat), Soilwater (VT), Soilwater+seawater, Spirulina Medium, TES-Astrephomene, TES-N/20, Trebouxia, Volvocacean, Volvocacean (3N), Volvox, Volvox-dextrose, Waris, Waris+SE and YT20); Culture Collection of Algae at the University of Göttingen, www.gwdg/de/˜epsag/Web/einstieg (i.e., Artificial Seawater Medium with Vitamins, Bacillariophycean Medium, Bacillariophycean Medium with Vitamins, Basal Medium, Basal Medium with 10% Euglena Medium and Vitamins, Basal Medium with Beef Extract, Basal Medium with H2SO4, Basal Medium with Peptone, Beggiatoa Medium, Bold's Basal Medium with triple Nitrate, Bold's Basal Medium with Vitamins, Bold's Basal Medium with Vitamins and triple Nitrate, Brackish Water Medium, Brackish Water Medium with Selenite, Brackish Water Medium with Silicate, Chilomonas Medium, Cyanidium Medium (=Acid Alga Medium), Cyanidium Medium+B1, Desmidiacean Medium, Desmidiacean Medium with Vitamin B1, Dunaliella acidophila Medium, Dunaliella Medium, Euglena Medium, Euglena Medium with Minerals, f/2 Medium, Half-strength Euglena Medium with Minerals, Kuhl-Medium for Unicellular Green Algae, Malt Peptone Medium, Modified Bold's Basal Medium, Modified Bold's Basal Medium for Heterotrophs, Ochromonas Medium, Polytoma Glucose, Polytoma Medium, Polytomella, Porphyridium Medium, Provasoli's enriched Seawater, Seawater Medium, Seawater Medium with Selenite, Seawater Medium with Silicate, Soil Water Media, Soil Water Medium with Barley (=GerstE), Soil Water Medium with CaCO3, Soil Water Medium with NH4MgPO4, Soil Water Medium with Pea, and Loamy Soil with Sand (=Erbs MS), Soil Water Medium with Pea (=ErbsE), Soil Water Medium with Pea and with Sand (=Erbs S), Soil Water Medium with Wheat, Spirulina Medium, TOM, Volvox Medium, WC Medium, WEES Medium and WeizenE); and Provasoli-Guillard National Center for the Culture of Marine Phytoplankton, ccmp.bigelow.org (i.e., Alkaline Soil Extract, CCMP's L/20 Derivatives, CCMP's L/20 Medium, CCMP's L/20/4+Va, DCM Medium, DYIV Medium (Freshwater), f/2 Medium, f/2 (35% SW), f/2* (75% SW), f/2*, f/2+Org, f/20-Si, f/20-Si+EDTA, f/2-Si, f/2-Si (75% SW), f/4 (50% SW), f/4-Si, f/50-Si, h/2 Medium, K Medium, L1 Medium, L1-Si, L1-Si (80% SW), Modified SN Medium (CCMP recipe), PC (Prochlorococcus) Medium, Prov Medium, Prov50, Prov50+Org, SN Medium, TBT Medium, and WCg Medium (Freshwater)).

E. Culture Variables

Table I below contains a non-limiting list of culture variables which define the incubation conditions that may be employed in the present invention in order to produce vigorous growth of the algae.

TABLE I
Culture Variables Affecting Growth

Temperature
Medium Composition
Agitation
Aeration
CO₂ bubbling
Light intensity
Illumination cycle
Illumination wavelength
Antibiotics
Organic solvents
Detergents
Physical Nature of Culture Vessel

Incubation of cultures may be performed at temperatures ranging from 2-100° C., depending on the individual strains, preferably from 10-30° C.

The culture medium employed is not critical to the present invention. Examples of such culture medium are shown above.

The amount of agitation to be used for the cultures can vary from no agitation to vigorous rotary or oscillatory shaking at as much as 4 cycles per second. Preferably, agitation is gentle (less than 1 cycle per second) or non-existent.

Aeration can be performed using, e.g., air, pure oxygen, or a mixture of oxygen and inert gas.

Similarly, CO₂ bubbling can be performed with pure CO₂ or with a mixture of CO₂ and either air or an inert gas. In either case, the preferable physical method for aeration is a gentle bubbling at less than 1 liter of gas per minute. However, more rapid bubbling, sparging, or other method of gas exchange at up to 5 liters of gas per minute can also be used.

Illumination cycles may be chosen from dark during 100% of the incubation, to light during 100% of the incubation.

Preferably, the illumination cycle consists of alternating 12 hour periods of light and dark.

The wavelength of light used for illumination can, e.g., range from 200 nanometer to 900 nanometers, and can be either a narrow spectrum or a mixture such as natural sunlight. Preferably, illumination contains a mix of wavelengths in the visible range, 400 to 700 nanometers. This intensity of the light can vary, e.g., from 0 to over 100 M⁻²sec⁻¹, preferably from 80 to 90 M⁻²sec⁻¹.

The particular antibiotic employed is not critical to the present invention. Examples of antibiotics which can be employed include penicillin and streptomycin at 100 units/ml and 100 μg/ml, respectively. In a preferred embodiment, no antibiotics are used.

The particular detergents employed is not critical to the present invention. Examples of detergents which can be employed include mild non-ionic detergents, such as Tween 20 or Nonidet, in concentrations less than 0.01% (v/v).

The particular culture vessel employed is not critical to the present invention and can have many geometries. For example, it may be a commercial bioreactor in a capillary, sheet, or tube configuration. The preferred culture vessel is an Ehrlenmeyer flask or a culture tube, made of a material transparent to visible light.

F. Further Subselection of Non-Prokaryotic Microalgae

Based on Interaction with the Precursor Compound In order to maximize the efficiency of obtaining biotransformations, after first eliminating those algae that do not grow in the presence of the solvent for the precursor compound, those algae whose metabolisms are unlikely to interact with the precursor compound are next eliminated. Specifically, a culture of each alga is grown for, e.g., a few days to as much as several weeks in the presence and absence of several different concentrations of the precursor compound (e.g., 5-500 μg/ml, preferably 20-150 μg/ml). Periodically, the cell concentration is determined to determine cell growth.

In one embodiment, the cell count is determined daily by counting in a haemocytometer in the presence or absence of a vital stain excluded from living cells, but not from dead cells. Other methods for determining cell growth can be used in the present invention, including but not limited to Coulter counting, light-scattering measurements, turbidity measurements, protein, DNA, or RNA concentration, or other methods familiar to one skilled in the art.

Strains that grow more slowly or more rapidly in the presence of precursor compound than in its absence are chosen for the further experimentation. Strains whose growth characteristics change in the presence of the precursor compound are presumed to be more likely to have an interaction between the precursor compound and the biochemical machinery of the cell than those whose growth is unaffected. Additionally, the highest concentration of precursor compound that allows for reasonable growth is preferably chosen for each strain in the subsequent step. Algae whose growth is unaffected are discarded, as are algal strains whose cell number decreases relative to the starting cell concentration.

G. Growth in the Presence of the Precursor Compound

Each algal strain of the panel further subselected as described above is grown in the presence of precursor compound using the conditions such as described in E above. In addition, each algal strain is grown in the absence of precursor compound as a growth control. Precursor compound is also dissolved in each culture medium at the same concentration as in each culture and incubated without algae under the same culture conditions (biotransformation control). In addition, cells may first be grown to stationary phase and then incubated with precursor compound with minimal cell division. After a period of incubation at 2-100° C., preferably 10-30° C., which can vary from a few days to several weeks, preferably 4-10 days, each culture is separated into a culture supernatant and cellular biomass. Any of a variety of well-known separation methods can be used to separate algal cells from culture supernatant. Those methods include but are not limited to filtration, centrifugation, and sedimentation.

H. Analysis and Purification

Each cell mass and/or supernatant may be extracted with an immiscible solvent, such as benzene or ethyl acetate to obtain a solvent extract containing the metabolite. In one embdodiment, the supernatant is not extracted and is used directly. The solvent extracts or supernatant may then be analyzed by a variety of techniques of analytical chemistry well known to those skilled in the art. Examples of applicable techniques of analytical organic chemistry include but are not limited to high pressure liquid chromatography, low pressure liquid chromatography, gas chromatography and thin-layer chromatography. The analysis result from each experimental sample can be compared to that of the corresponding growth control and biotransformation control to discover signals from molecules present in the experimental sample, but not in the corresponding growth control or biotransformation control samples.

In addition, the solvent extracts or supernatant may optionally be treated by a number of methods of analytical organic chemistry to partially or completely purify the metabolite. The particular purification method employed is not critical to the present invention. Examples of such purification methods include high pressure liquid chromatography, low pressure liquid chromatography, phase partitioning, and gas chromatography, either singly or in combination.

The preferred metabolite candidates can then be identified using methods of organic chemistry, such as mass spectroscopy (MS) or nuclear magnetic resonance (NMR), that are well-known to those skilled in the art of analytical organic or bioanalytical chemistry for structural analysis.

The following examples are provided for illustrative purposes only, and are in no way intended to limit the scope of the present invention.

### EXAMPLE 1

This example demonstrates biotransformation of the precursor compound, (S)-(−)-3-(Benzyloxycarbonyl)-4-oxazolidinecarboxylic acid, into

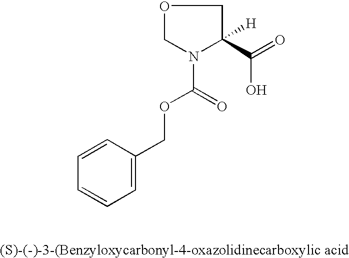

the metabolite (S)-(−)-3-(Benzyloxycarbonyl)-1-amino-2-hydroxycarboxylic

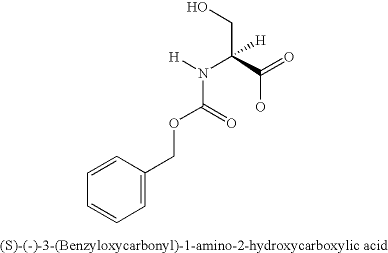

acid. (S)-(−)-3-(Benzyloxycarbonyl)-4-oxazolidinecarboxylic acid is a useful building block for the synthesis of β-lactam antibiotics, as well as a useful starting reagent for the synthesis of many chiral compounds by diastereoselective Michael additions and Dies-Alder reactions. Its metabolite is a protected chiral α-amino acid, and so it is also a useful starting point for many other chiral syntheses (Ager et al, Chem. Rev., 96:835 (1996)).

(A) Non-Prokaryotic Microalgae Strains

The initial panel of microalgae consisted of the strains listed in Table II below. The characteristics of evolutionary, ecological and metabolic diversity of these strains are shown in Table II below. The panel included representatives from classes Trebouxiophyceae, Chlorophyceae, Cryptomonideae, Euglenophyta, Raphidophyceae, Diatomatideae, Prasinophyceae, . . . . Ecological niches included but are not limited to marine (including benthic, epiphytic and planktonic, near shore and open ocean, brackish water and halophilic, tropic and temperate); freshwater benthic, epiphytic, and planktonic (including alkaline creek, eutrophic lakes and ponds, oligotrophic lakes and ponds, alpine lakes and ponds); and non-aquatic (temperate soil, tropical soil, cold soil and air-borne). Types of metabolism included photoautotrophs, heterotrophs and mixotrophs.

TABLE II
Initial Panel of Microalgae

| Algal Strain | Taxonomic Affinity | Ecological Habitat |
|---|---|---|
| Amphiprora palludosa | Phylum Ochrophyta A Diatom | Marine and brackish water, planktonic |
| Amphora coffeaeformis | Phylum Ochrophyta, Class Bacillariophyceae | Marine and brackish water |
| Anksitrodesmus angustus | Phylum Chlorophyta, Class Chlorophyceae | Soil |
| Ankyra starii | Phylum Chlorophyta, Class Chlorophyceae | Soil |
| Aphanochaete elegans | Phylmn Chlorophyta, Class Chlorophyceae | Stagnant water, epiphytic |
| Asterococcus superbus | Phylum Chlorophyta, Class Chlorophyceae | Boggy pools |
| Axilococcus clirgmanii | Phylum Chlorophyta, Class Chlorophyceae | Soil |
| Axilosphaera vegetata | Phylum Chlorophyta, Class Chlorophyceae | Soil |
| Borodinellopsis texensis | Phylum Chlorophyta, Class Chlorophyceae | Soil |
| Botrydiopsis arhiza | Phylum Ochrophyta, Class Xanthophyceae | Pond |
| Botrydiopsis aspina | Phylum Ochrophyta, Class Xanthophyceae | Soil |
| Botrydium becherianum | Phylum Ochrophyta, Class Xanthophyceae | Soil |
| Botrydium cystosum | Phylum Ochrophyta, Class Xanthophyceae | Marine |
| Botryococcus braunii | Phylum Chlorophyta, Class Chlorophyceae | Freshwater and soil |
| Brachiomanas submarina | Phylum Chlorophyta, Class Chlorophyceae | Cold water lakes and ponds |
| Bracteacoccus cinnibarinus | Phylum Chlorophyta, Class Chlorophyceae | Soil |
| Bumilleria exilis | Phylum Ochrophta, Class Xanthophyceae | Cold soil |
| Bumilleriopsis filiformis | Phylum Ochrophta, Class Xanthophyceae | Soil |
| Cephalaleuros parasitiaus | Phylum Chlorophyta, Class Chlorophyceae | Leaf parasite |
| Carteria eugametos | Phylum Chlorophyta, Class Chlorophyceae | Freshwater |
| Chamaetrichon capsulatum | Phylum Chlorophyta, Class Chlorophyceae | Freshwater |
| Characium astipitatum | Phylum Chlorophyta, Class Chlorophyceae | Soil |
| Characium californicum | Phylum Chlorophyta, Class Chlorophyceaee | Freshwater epiphytic or benthic |
| Chlamydomonas reinhardtii | Phylum Chlorophyta, Class Chlorophyceae | Soil |
| Chlorella minutisima | Phylum Chlorophyta, Class Trebouxiophyceae | Stagnant water, tolerating broad |
| | | salinity range. |
| Chlorellidium tetrabotrys | Phylum Ochrophta, Class Xanthophyceae | Soil |
| Chloridella neglecta | Phylum Ochrophta, Class Xanthophyceae | Soil |
| Chlorochytrium lemnae | Phylum Chlorophyta, Class Chlorophyceae | Freshwater |
| Chlorocloster engadines | Phylum Ochrophta, Class Xanthophyceae | Soil |
| Chlorococcum acidum | Phylum Chlorophyta, Class Chlorophyceae | Soil |
| Chlorococcum texasum | Phylum Chlorophyta, Class Chlorophyceae | Soil |
| Chlorogonium elongatum | Phylum Chlorophyta, Class Chlorophyceae | Wet soil |
| Chloromonas rosae | Phylum Chlorophyta, Class Chlorophyceae | Soil, “snow alga” |
| Chlorosarcina longispinosa | Phylum Chlorophyta, Class Chlorophyceae | Soil |
| Chlorosarcinopsis | Phylum Chlorophyta, Class Chlorophyceae | Soil, colonial |
| auxotrophica | | |
| Chroomonas pochmani | Phylum Crytophyta, Class Cryptophyceae | Marine |
| Chrysochromulina chiton | Phylum Prymnesiophyta, Class | Marine |
| | Prymnesiophyceae | |
| Cryptochrysis rubens | Phylum Crytophyta, Class Cryptophyceae | Marine, planktonic |
| Cryptomonas ovata | Phylum Crytophyta, Class Cryptophyceae | Planktonic in oligotrophic lakes |
| Ctenocladus circinnatus | Phylum Chlorophyta, Class Ulvophyceae | Brackish soil |
| Euglena geniculate | Euglenophyceae | Soil |
| Euglena gracilis | Phylum Euglenophyta | Fresh water with high organic content |
| Moromastix sp. | Phylum Crytophyta, Class Cryptophyceae | Cold freshwater, planktonic |
| Olithodiscus sp. | Phylum Ochrophyta, Class Raphidophyceae | Marine, planktonic |
| Phacus caudata | Euglenophyta | Soil |
| Phaeodactylum tricornutum | Phylum Ochrophyta A Diatom | Marine epiphytic |
| Pinnularia sp. | Phylum Ochrophyta, Class Bacillariophyceae | Tide pools |
| Platymonas sp. | Phylum Chlorophyta, Class Prasinophyceae | Marine, high organic content, |
| | | planktonic |
| Porphyridium cruentum | Phylum Rhodophyta, Class Bangiophycidae | Soil |
| Prasinocladus sp. | Phylum Chlorophyta, Class Prasinophyceae | Near-shore marine planktonic |
| Prymnesium sp. | Phylum Prymnesiophyta, Class | Marine |
| | Prymnesiophyceae | |
| Rhodomonas sp. | Phylum Crytophyta, Class Cryptophyceae | |
| Scenedesmus obliquus | Phylum Chlorophyta, Class Chlorophyceae | Freshwaters |
| Skeletonema costatum | Phylum Ochrophyta, Class Bacillariophyceae | Marine diatom |
| Tetraselmis chuii | Phylum Chlorophyta, Class Prasinophyceae | Marine |

(B) Subselection Based on Growth Characteristics in the Presence of Solvent

15 ml cultures of each microalgae shown in Table II above were grown under the various conditions shown in Table III below in the presence and absence of 0.4% (v/v) methanol as the solvent for the precursor compound, (S)-(−)-3-(benzyloxycarbonyl)-4-oxazolidinecarboxylic acid. Several strains of algae were strongly affected by solvent, i.e., the cell count of the culture actually decreased over time or visual inspection revealed large numbers of dead or dying cells compared to controls. These strains were judged unsuitable for biotransformation. Others had unsuitable growth characteristics, such as extremely slow growth or contaminating co-culturing bacteria. These strains were discarded to produce the subset of strains shown in Table III below.

TABLE III
Panel After Growth Optimization and Culture Conditions

| *Algal Strain | Culture Conditions |
|---|---|
| Amphiprora palludosa | DAS medium, 22° C. on |
| | a rotary shaker (100 gyrations/min) with |
| | a 12-hr on/12-hr off photoperiod |
| Bracteacoccus cinnibarinus | MVM medium, 20° C. with |
| | a 12-hr on/12-hr off photoperiod |
| Characium californicum | MVM medium, 22° C. on |
| | a rotary shaker (100 gyrations/min) with |
| | a 12-hr on/12-hr off photoperiod |
| Chlamydomonas reinhardtii | TAP medium, 22° C. on |
| | a rotary shaker (100 gyrations/min) with |
| | a 12-hr on/12-hr off photoperiod |
| Chlorella minutisima | ERD medium, 22° C. on |
| | a rotary shaker (100 gyrations/min) with |
| | a 12-hr on/12-hr off photoperiod |
| Chloromonas rosae | MVM medium, 22° C. on |
| | a rotary shaker (100 gyrations/min) with |
| | a 12-hr on/12-hr off photoperiod |
| Chlorosarcinopsis | MVM medium, 22° C. on a |
| auxotrophica | rotary shaker (100 gyrations/min) with |
| | a 12-hr on/12-hr off photoperiod |
| Cryptocrysis rubens | ERD medium, 22° C. on |
| | a rotary shaker (100 gyrations/min) with |
| | a 12-hr on/12-hr off photoperiod |
| Cryptomonas ovata | INV medium, 20° C. with |
| | a 12-hr on/12-hr off photoperiod |
| Euglena gracilis | Old Euglena medium, 22° C. |
| | on a rotary shaker (100 gyrations/min) |
| | with a 12-hr on/12-hr off photoperiod |
| Moromastix sp. | INV medium, 20° C. with |
| | a 12-hr on/12-hr off photoperiod |
| Olithodiscus sp. | ERD medium, 22° C. on |
| | a rotary shaker (100 gyrations/min) with |
| | a 12-hr on/12-hr off photoperiod |
| Phaeodactylum tricornutum | DAS medium, 22° C. on |
| | a rotary shaker (100 gyrations/min) with |
| | a 12-hr on/12-hr off photoperiod |
| Platymonas sp. | DAS medium, 22° C. on |
| | a rotary shaker (100 gyrations/min) with |
| | a 12-hr on/12-hr off photoperiod |
| Prasinocladus sp. | DAS medium, 22° C. on |
| | a rotary shaker (100 gyrations/min) with |
| | a 12-hr on/12-hr off photoperiod |
| Scenedesmus obliquus | TAP medium, 22° C. |
| | on a rotary shaker (100 gyrations/min) with |
| | a 12-hr on/12-hr off photoperiod |

*Almost all of the strains were cultured at 22° C. on a rotary shaker (100 gyrations/min) with a 12-hr on/12-hr off photoperiod. A few strains (Moromastix sp., C. ovata and B. cinnibarinus) were cultured under similar conditions, except that they were not agitated and were kept at 20° C. None of the strains were grown in CO₂-enriched medium.

(C) Further Subselection Based on Each Strain's Interaction with the Precursor Compound

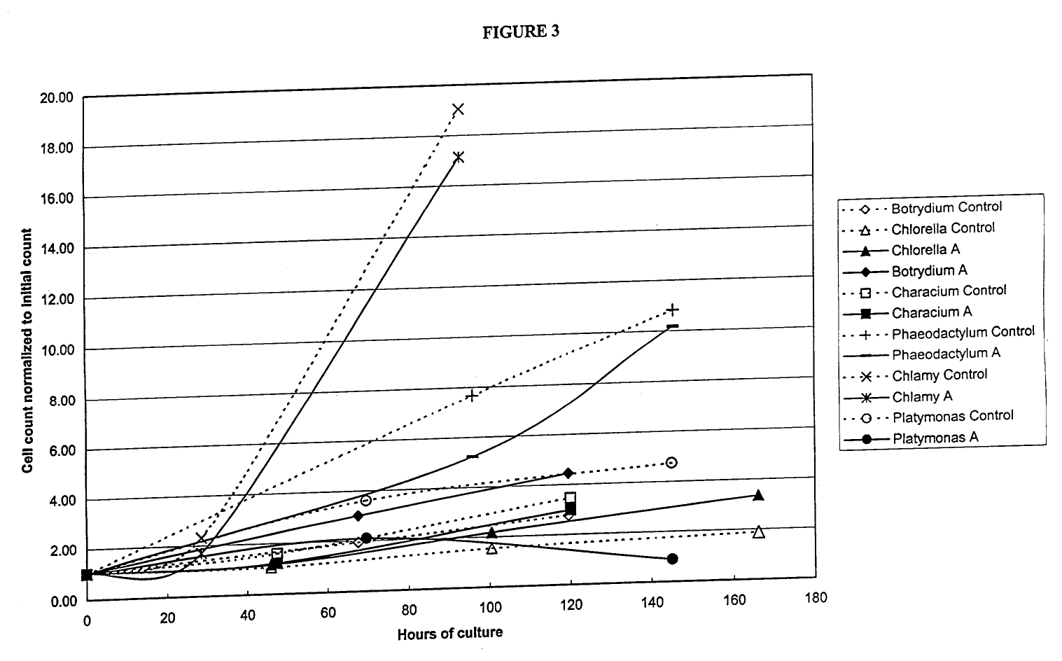

Three 15 ml cultures of each of the microalgae listed in Table III above were grown in 30 ml flasks. Growth control cultures contained algae and medium only. Growth control cultures in the presence of solvent contained algae, medium, and 0.06 ml of methanol, the solvent chosen for the precursor compound. Experimental cultures contained algae, medium, and 0.06 ml of a 25 mg/ml solution of (S)-(−)-3-(Benzyloxycarbonyl)-4-oxazolidinecarboxylic acid (precursor compound) dissolved in methanol, yielding a final concentration of 100 μg/ml. The cultures were incubated under the conditions shown in Table III above. Cell counts were performed using a haemocytometer on days 0, 2, 4 and 7. A selection of the results are shown in FIG. 3.

In some cases, the precursor compound affected growth strongly, so that the cell concentration increased little over that of either growth control during the course of the experiment or actually decreased over time, indicating that the precursor compound was causing cell death. The test with Platymonas shown in FIG. 3 is an example of precursor compound causing cell death. Microalgal strains that showed increased growth relative to the growth controls (for example, Chlorella and Botrydium in FIG. 3) or that showed moderate, but not severe decreases in growth relative to the growth controls (for example Chlamydomonas, and Pheodactylum in FIG. 3) were chosen for the final panel. Table IV below lists those microalgae showing an effect on growth by the precursor compound.

TABLE IV
Final Algal Panel

| | |
|---|---|
| Amphiprora palludosa | Cryptomonas ovata |
| Bracteacoccus cinnibarinus | Euglena gracilis |
| Characium californicum | Moromastix sp. |
| Chlamydomonas reinhardtii | Olithodiscus sp. |
| Chlorella minutisima | Phaeodactylum tricornutum |
| Chloromonas rosae | Platymonas sp. |
| Chlorosarcinopsis auxotrophica | Prasinocladus sp. |
| Cryptocrysis rubens | Scenedesmus obliquus |

(D) Growth in the Presence of Precursor Compound

Table V below lists each microalgae in the final panel, its starting cell concentration, the concentration of precursor compound in the growth medium, the starting and final cell count, and the method of separation of the resulting biomass from the culture supernatant. The final panel of algal strains were grown for 7 days under the conditions listed in Table III above in the presence of the precursor compound prior to separation of the resulting biomass from the culture supernatant. Cell counts were not determined in every case for the strains listed in Table V.

TABLE V
Final Biotransformation Conditions

| | Concentration | | | |
|---|---|---|---|---|
| | of Precursor | Starting Cell | Final Cell | Separation |
| Algae | Compound | Concentration | Concentration | Method |
| Amphiprora palludosa | 100 μg/ml | Not determined | Not determined | Centrifugation |
| Bracteacoccus | 100 μg/ml | Not determined | Not determined | Centrifugation |
| cinnibarinus | | | | |
| Characium | 200 μg/ml | Not determined | Not determined | Centrifugation |
| californicum | | | | |
| Chlamydomonas | 200 μg/ml | 2.2 × 10⁷ | 1.58 × 10⁹ | Centrifugation |
| reinhardtii | | | | |
| Chlorella minutisima | 100 μg/ml | 1.52 × 10⁶ | 1.08 × 10⁷ | Centrifugation |
| Chloromonas rosae | 100 μg/ml | 1.75 × 10⁵ | 1.13 × 10⁶ | Centrifugation |
| Chlorosarcinopsis | 200 μg/ml | Not determined | Not determined | Centrifugation |
| auxotrophica | | | | |
| Cryptocrysis rubens | 200 μg/ml | Not determined | 3.09 × 10⁶ | Centrifugation |
| Cryptomonas ovata | 200 μg/ml | Not determined | 1.3 × 10⁵ | Centrifugation |
| Euglena gracilis | 100 μg/ml | Not determined | 6.6 × 10⁵ | Centrifugation |
| Moromastix sp. | 200 μg/ml | 6.16 × 10⁵ | 7.4 × 10⁵ | Centrifugation |
| Olithodiscus sp. | 50 μg/ml | 1.09 × 10⁶ | 1.3 × 10⁶ | Centrifugation |
| Phaeodactylum | 200 μg/ml | 1.18 × 10⁶ | 4.08 × 10⁶ | Filtration |
| tricornutum | | | | |
| Platymonas sp. | 100 μg/ml | Not determined | Not determined | Filtration |
| Prasinocladus sp. | 100 μg/ml | Not determined | Not determined | Centrifugation |
| Scenedesmus obliquus | 200 μg/ml | 6.38 × 10⁶ | 2.12 × 10⁷ | Centrifugation |

(E) Purification, Analysis and Identification

In the present example, culture supernatants were not extracted with an organic solvent. However, it is possible to extract the supernatants by, for example, adding an equal volume of an organic solvent that is not miscible with water, mixing well to emulsify the mixture, and then separating the two liquid phases by centrifugation or in a separatory funnel or by some other means. Examples of suitable organic solvents include but are not limited to benzene and ethyl acetate.

The culture supernatants were analyzed on a Hewlett Packard High Pressure Liquid Chromatograph (HPLC), Model 1100 or 1050, equipped with solvent delivery system, solvent degasser, temperature controlled column compartment, sample auto injector and diode array detector. In addition, the Model 1100 has a mass selective detector. The column used was a Hewlitt Packard Zorbax Eclipse XDB-C8, 5 micron, 4.6 mm×150 mm. The flow rate was 1.0 ml per minute. 5.0 μl of extract aliqots were injected and the column was developed with the gradient shown in Table VI below.

TABLE VI
HPLC Elution Conditions for Analysis of Culture Supernatants

| Time | % of 0.1% (v/v) | % (v/v) of |
|---|---|---|
| (minutes) | Phosphoric Acid | Acetonitrile |
| 0.00 | 65 | 35 |
| 5.00 | 65 | 35 |
| 5.50 | 55 | 45 |
| 12.00 | 55 | 45 |
| 15.00 | 65 | 35 |

The eluate was analyzed at 210 nm with a bandwidth of 8 nm. Each chromatograph of culture supernatant was compared to chromatographs of culture growth control and of biotransformation control supernatants. Peaks that appeared in the chromatographs of supernatants of cultures incubated with algae and precursor compound, but not in chromatographs of control samples, were considered candidates for modified precursor compound (metabolite), and were selected for further study. Of the 16 strains tested, 5 strains, i.e., Amphipora, Cryptochysis, Chlamydomonas, Scenedesmus, and Cryptomonas, each had one candidate peak. The elution time for the candidate peaks from Chlamydomonas, and Scenedesmus were almost identical, which is believed to indicate identical transformations of the precursor.

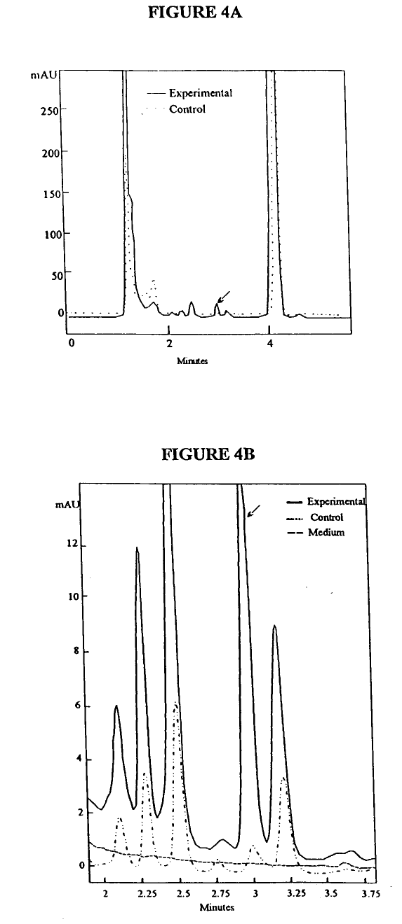

FIG. 4A shows a representative HPLC chromatograph using the solvent extract from Chlamydomonas with a candidate peak marked. FIG. 4B shows the area of the HPLC chromatogram around the marked peak in FIG. 4A (shown at an increased resolution).

The peak marked in FIG. 4B was subjected to further analysis by mass spectroscopy (MS). The medium was analyzed by combined liquid chromatography and mass spectroscopy (LC/MS) using a Phenomenex Luna 3 micro phenyl-hexyl column, 150 mm×2.0 mm. The column was developed at 30° C., at 0.40 ml per minute with the solvent program shown in Table VII below.

TABLE VII
LC/MS Elution Conditions for the
Analysis of Candidate HPLC Peaks by MS

| Elaped | % of 0.1% (v/v) | % (v/v) of | |
|---|---|---|---|
| Time | Formic Acid | Acetonitrile | Curve Type |
| 0.00 | 90 | 10 | — |
| 10.00 | 20 | 80 | Linear |
| 12.00 | 20 | 80 | Hold |
| 13.00 | 90 | 10 | Linear |
| 18.00 | 90 | 10 | Re-equilibration |

For mass spectroscopy, the ionization mode was ESP positive, with a cone voltage of 25 volts. The mass range was 60 to 500 amu, the source temperature was 150° C., and the time window was 0 to 15 minutes. The ESI nebulizer gas was nitrogen at 20 l/hr, and the bath gas was also nitrogen but at 350 l/hr. The mass spectrometer was a Fisons VG Quattro.

The precursor compound eluted at 7.63 minutes and had a mass of 252. The candidate peak eluted at 7.58 minutes and had a mass of 240.

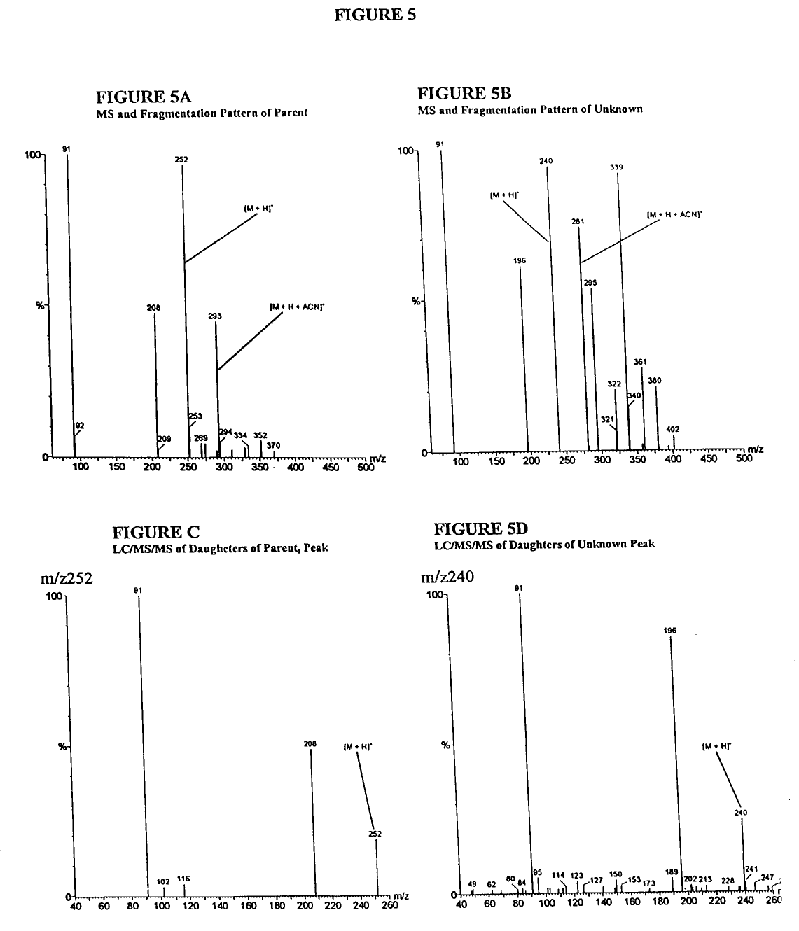

In order to determine the structure of the putative metabolite, liquid chromatography coupled to 2-dimensional mass spectroscopy (LC/MS/MS) was performed on the material from the HPLC peak marked in FIG. 4B as well as on the parent molecule to analyze daughter fragments of the precursor and candidate metabolites. LC/MS/MS was performed under the same conditions as the LC/MS, except that the detector wavelength was 210 nm, the collision gas was argon and the collision energy was 7 eV. The results were analyzed using MassLynx Version 3.4 (Micromass) software. The results are shown in FIGS. 5A-5D.

As shown in FIGS. 5A and 5C, the parent molecule has a protonated mass of 252 and a mass for the adduct between the protonated parent and acetonitrile of 293. It has major fragments of masses 91 and 208. As shown in FIGS. 5B and 5D, the unknown has a protonated mass of 240, giving it a mass difference of 12 from the parent. It has major fragments of masses 91 and 196. The fragment of the unknown at 196 has a mass of 12 less that the major fragment in the parent of mass 208, indicating that this fragment contains the transformation. The fragment of mass 91 is present in both, indicating that it is derived from a portion of the molecule that was not modified in the unknown.

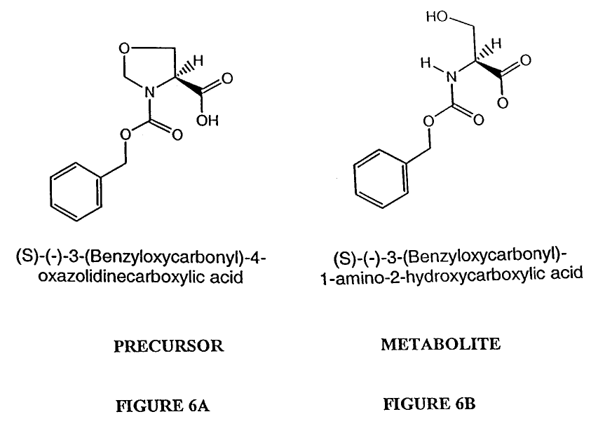

FIG. 6A shows the structure of the precursor compound and FIG. 6B shows the predicted structure of the metabolite deduced from the fragmentation patterns shown in FIG. 5. It is apparent that in this particular biotransformation, a one carbon fragment is excised from the oxazolidine ring, opening the ring and resulting in the alcohol.

### EXAMPLE 2

This example demonstrates biotransformation of a second precursor compound, tert-butyl[S-(R*-R*)]-(−)-(1-oxiranyl-2-phenylethylcarbamate):

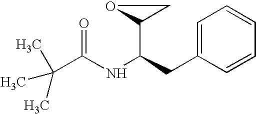

tert-butyl[S-(R*-R*)]-(−)-(1-oxiranyl-2-phenylethylcarbamate), hereinafter referred to as Precursor 2, is a pivotal building block for the synthesis of hydroxyethylamine dipeptide isosteres, which class includes but is not limited to many HIV protease inhibitors.

Precursor 2 was biotransformed by the addition of a cysteine moiety into one of two possible sites, to yield either the hypothesized metabolite N1-(tert-butoxycarbonyl)-N1-[1-phenyl(2,3-dihydroxypropyl)methyl]cysteinamide

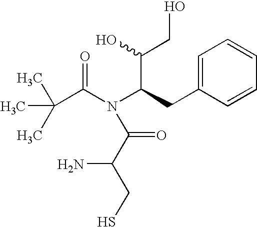

5 or the hypothesized metabolite S-{2-hydroxy-3-[(tert-butoxycarbonyl)amino]-4-phenylbutyl}cysteine.

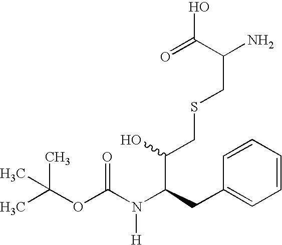

The derivatized cysteinamide metabolite contains 2 mirror image peptide bonds sharing the same nitrogen atom and is therefore an excellent synthesis route for potential symmetric inhibitors of homodimeric proteins. Some closely related compounds of the amino diols class have been shown to have anti-HIV activity (Chen et al, J. Med. Chem., 39(10):1991-2007 (1996)).

S-{2-hydroxy-3-[(tert-butoxycarbonyl)amino]-4-phenylbutyl}cysteine is a non-proteinogenic amino acid, an amino acid that is not used in nature to synthesize proteins. Non-proteinogenic amino acids are useful for the synthesis of inhibitors of specific protein-protein interactions. Common strategies for blocking such interactions include the design of a peptide that mimics, but is not identical, to the substrate binding site, such as one containing a non-proteinogenic amino acid. Also, non-proteinogenic derivatives of cysteine are believed to be useful as inhibitors of the cysteine proteases (Albeck et al, Biochem. J, 346:71-76 (2000)). Both of these inhibitory compound classes are useful to the pharmaceutical and agrichemical industries. In general, such compounds are synthesized by complex processes or, more recently, by fermentation using a genetically modified strain of E. coli (U.S. Patent Publication 2002/0039767).

S-{2-hydroxy-3-[(tert-butoxycarbonyl)amino]-4-phenylbutyl}cysteine contains a motif which is shared by the cysteinyl leukotrienes (LTs), C₄, D₄, E₄ and F₄. The cysteinyl leukotrienes are components of the slow-reacting substance of anaphylaxis (Hammarstrom et al, J. Biochem. Biophys. Res. Commun., 92:946 (1980)) and have been strongly implicated in the aetiology of asthma (Lam et al, Am. J. Respir. Crit. Care Med., 161(2): S16-S19 (2000)). LTs are also believed to promote cellular chemotaxis toward sites of tissue injury (Delgado et al, supra).

A cysteinyl LT is shown below. R may be either H or glycine, and R′ may be either H or γ-glutamic acid.

The shared motif is shown below. R and R′ are as described above, Leukotrienes

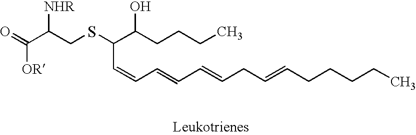

and R″ and R′″ are unspecified chemical groups.

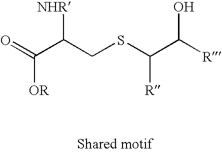

The cysteinyl leukotrienes are synthesized in nature by a condensation between glutathione and the epoxide ring on leukotriene A₄ (Delgado et al, supra; and Lam et al, Am. J. Respir. Crit. Care Med., 161(2):S16-S19 (2000)). The enzyme responsible for the reaction, LTC₄ synthase, is an example of a glutathione S-transferase (GSH transferase). LTC₄ synthase is very specific in its substrate affinity, and recognizes only glutathione to produce LTC₄. The other cysteinyl leukotrienes are produced by selective cleavage of the glutathione moiety of LTC₄ (Ibid.). Additionally, LTC₄ synthase is different from other known glutathione S-transferases in that its substrate is an epoxide moiety which is opened during the reaction. An enzyme capable of directly conjugating cysteine instead of glutathione to LTA₄ would produce LTE₄ in one step as opposed to three steps (conjugation with glutathione followed by two hydrolytic cleavages). In addition, such an enzyme would be useful in the synthesis of cysteinyl leukotriene analogues as potential LT membrane receptor blockers to be used, for example, in the treatment of asthma as well as other diseases and disorders.

(A) Microalgae Strains

The initial panel of microalgae consisted of the strains listed in Table II above. The characteristics of taxonomic and ecological diversity of these strains are also shown in Table II. The panel included representatives from classes Trebouxiophyceae, Chlorophyceae, Cryptomonideae, Euglenophyta, Raphidophyceae, Diatomatideae, Prasinophyceae, . . . . Ecological niches included but were not limited to marine (including benthic, epiphytic and planktonic, near shore and open ocean, brackish water and halophilic, tropic and temperate); freshwater benthic, epiphytic, and planktonic (including alkaline creek, eutrophic lakes and ponds, oligotrophic lakes and ponds, alpine lakes and ponds); and non-aquatic (temperate soil, tropical soil, cold soil and air-borne). Types of metabolism included photoautotrophs, heterotrophs and mixotrophs.

(B) Subselection Based on Growth Characteristics in the Presence of Solvent

15 ml cultures of each microalga shown in Table II above was grown under the various conditions shown in Table III above in the presence and absence of 0.4% (v/v) methanol as the solvent for Precursor 2, tert-butyl[S-(R*-R*)]-(−)-(1-oxiranyl-2-phenylethylcarbamate). As in Example 1, several strains of algae were strongly affected by solvent such that the cell count of the culture actually decreased over time or visual inspection revealed large numbers of dead or dying cells compared to controls. These strains were judged unsuitable for biotransformation. Others had unsuitable growth characteristics, such as extremely slow growth or contaminating co-culturing bacteria. These strains were discarded to produce the subset of strains shown in Table VIII.

TABLE VIII
Panel After Growth Optimization and Culture Conditions

| *Algal Strain | Culture Conditions |
|---|---|
| Amphiprora palludosa | DAS medium, 22° C. on a |
| | rotary shaker (100 gyrations/min) with |
| | a 12-hr on/12-hr off photoperiod |
| Ankistrodesmus angustus | MVM medium, 20° C. with |
| | a 12-hr on/12-hr off photoperiod |
| Botrydium becherianum | MVM medium, 20° C. with |
| | a 12-hr on/12-hr off photoperiod |
| Bracteacoccus cinnibarinus | MVM medium, 20° C. with |
| | a 12-hr on/12-hr off photoperiod |
| Characium californicum | MVM medium, 22° C. on |
| | a rotary shaker (100 gyrations/min) with |
| | a 12-hr on/12-hr off photoperiod |
| Chlamydomonas reinhardtii | TAP medium, 22° C. on |
| | a rotary shaker (100 gyrations/min) with |
| | a 12-hr on/12-hr off photoperiod |
| Chlorella minutisima | ERD medium, 22° C. on |
| | a rotary shaker (100 gyrations/min) with |
| | a 12-hr on/12-hr off photoperiod |
| Chloromonas rosae | MVM medium, 22° C. on |
| | a rotary shaker (100 gyrations/min) with |
| | a 12-hr on/12-hr off photoperiod |
| Chlorosarcinopsis auxotrophica | MVM medium, 22° C. on |
| | a rotary shaker (100 gyrations/min) with |
| | a 12-hr on/12-hr off photoperiod |
| Cryptocrysis rubens | ERD medium, 22° C. on |
| | a rotary shaker (100 gyrations/min) with |
| | a 12-hr on/12-hr off photoperiod |
| Cryptomonas ovata | INV medium, 20° C. with |
| | a 12-hr on/12-hr off photoperiod |
| Euglena gracilis | Old Euglena medium, 22° C. on |
| | a rotary shaker (100 gyrations/min) |
| | with a 12-hr on/12-hr off photoperiod |
| Moromastix sp. | INV medium, 20° C. with |
| | a 12-hr on/12-hr off photoperiod |
| Olithodiscus sp. | ERD medium, 22° C. on |
| | a rotary shaker (100 gyrations/min) with |
| | a 12-hr on/12-hr off photoperiod |
| Phaeodactylum tricornutum | DAS medium, 22° C. on |
| | a rotary shaker (100 gyrations/min) with |
| | a 12-hr on/12-hr off photoperiod |
| Platymonas sp. | DAS medium, 22° C. on |
| | a rotary shaker (100 gyrations/min) with |
| | a 12-hr on/12-hr off photoperiod |
| Prasinocladus sp. | DAS medium, 22° C. on |
| | a rotary shaker (100 gyrations/min) with |
| | a 12-hr on/12-hr off photoperiod |
| Scenedesmus obliquus | TAP medium, 22° C. on |
| | a rotary shaker (100 gyrations/min) with |
| | a 12-hr on/12-hr off photoperiod |

*Almost all of the strains were cultured at 22° C. on a rotary shaker (100 gyrations/min) with a 12-hr on/12-hr off photoperiod. A few strains (Moromastix sp., C. ovata and B. cinnibarinus) were cultured under similar conditions, except that they were not agitated and were kept at 20° C. None of the strains was grown in CO₂-enriched medium.

(C) Further Subselection Based on Each Strain's Interaction with Precursor 2

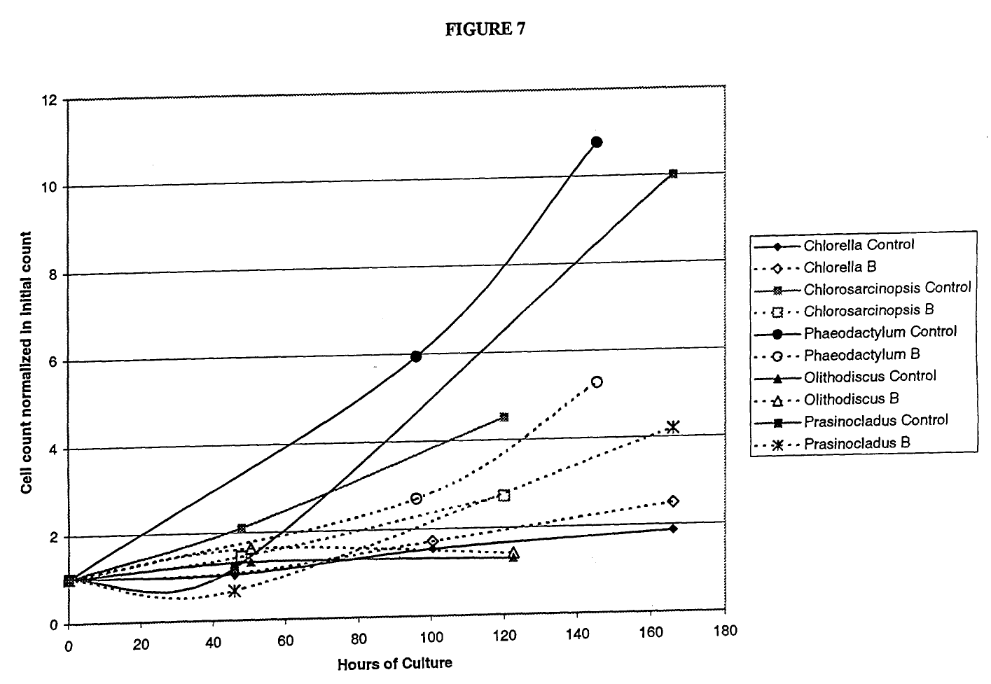

Three 15 ml cultures of each of the microalgae listed in Table VIII above were grown in 30 ml flasks. Growth control cultures contained algae and medium only. Growth control cultures in the presence of solvent contained algae, medium, and 0.06 ml of methanol, the solvent chosen for Precursor 2. Experimental cultures contained algae, medium, and 0.06 ml of a 25 mg/ml solution of tert-butyl[S-(R*-R*)]-(−)-(1-oxiranyl-2-phenylethylcarbamate) (precursor compound) dissolved in methanol, yielding a final concentration in the culture medium of 100 μg/ml. The cultures were incubated under the conditions shown in Table VIII above. Cell counts were performed using a haemocytometer on days 0, 2, 4 and 7. A selection of the results is shown in FIG. 7.

In some cases, Precursor 2 affected the growth very little. For example, FIG. 7 shows that the growth of Olisthodiscus was not significantly affected by Precursor 2. In other cases, Precursor 2 decreased the growth rate of the cells. FIG. 7 shows that, for example, Prasinocladus, Phaeodactylum, and Chlorosarcinopsis grew more slowly in the presence of precursor than in its absence. In a few cases, the growth rate of algae actually increased in the presence of precursor relative to controls. In FIG. 7, the growth of Chlorella is faster in the presence of this precursor than in control cultures. It may be noted, by way of contrast, that, as shown in FIG. 4, the growth of Chlorella was negatively affected by Precursor 2 used in Example 1 (see above). Those strains of algae showing changes in growth rate (other than severe decreases in growth relative to the growth controls) were chosen for the final panel. Table IX below lists those microalgae in the final panel.

TABLE IX
Final Algal Panel

| | |
|---|---|
| Amphiprora palludosa | Chlorosarcinopsis auxotrophica |
| Bracteacoccus cinnibarinus | Cryptomonas ovata |
| Characium californicum | Euglena gracilis |
| Chlamydomonas reinhardtii | Phaeodactylum tricornutum |
| Chlorella minutisima | Platymonas sp. |
| Chloromonas rosae | Scenedesmus obliquus |

(D) Growth in the Presence of Precursor Compound

Table X below lists each microalgae in the final panel, its starting cell concentration, the concentration of precursor compound in the growth medium, the starting and final cell count, and the method of separation of the resulting biomass from the culture supernatant. The final panel of algal strains was grown for 7 days under the conditions listed in Table VIII above in the presence of Precursor 2 prior to separation of the resulting biomass from the culture supernatant. Cell counts were not determined in every case for the strains listed in Table X.

TABLE X
Final Biotransformation Conditions

| | Concentration | | | |
|---|---|---|---|---|
| | of Precursor | Starting Cell | Final Cell | Separation |
| Algae | Compound | Concentration | Concentration | Method |
| Amphiprora palludosa | 100 μg/ml | Not determined | Not determined | Centrifugation |
| Bracteacoccus | 100 μg/ml | Not determined | Not determined | Centrifugation |
| cinnibarinus | | | | |
| Characium | 100 μg/ml | Not determined | Not determined | Centrifugation |
| californicum | | | | |
| Chlamydomonas | 100 μg/ml | 1.3 × 10⁶ | 4.98 × 10⁶ | Centrifugation |
| reinhardtii | | | | |
| Chlorella minutisima | 100 μg/ml | 1.56 × 10⁶ | 6.62 × 10⁶ | Centrifugation |
| Chloromonas rosae | 100 μg/ml | Not determined | Not determined | Centrifugation |
| Chlorosarcinopsis | 200 μg/ml | 1.15 × 10⁵ | 5.87 × 10⁵ | Centrifugation |
| auxotrophica | | | | |
| Cryptomonas ovata | 100 μg/ml | Not determined | 8.5 × 10⁴ | Centrifugation |
| Euglena gracilis | 100 μg/ml | Not determined | 6.25 × 10⁵ | Centrifugation |
| Olithodiscus sp. | 50 μg/ml | 1.10 × 10⁶ | 8.57 × 10⁵ | Centrifugation |
| Phaeodactylum | 100 μg/ml | 1.44 × 10⁶ | 2.58 × 10⁶ | Filtration |
| tricornutum | | | | |
| Platymonas sp. | 100 μg/ml | Not determined | Not determined | Filtration |
| Prasinocladus sp. | 100 μg/ml | Not determined | Not determined | Centrifugation |
| Scenedesmus obliquus | 100 μg/ml | 5.75 × 10⁶ | 1.2 × 10⁷ | Centrifugation |

(E) Purification, Analysis and Identification

(1) Stability Studies

Chromatography conditions for analysis of Precursor 2 were developed for the Hewlett Packard High Pressure Liquid Chromatograph (HPLC), Model 1100 or 1050, equipped with solvent delivery system, solvent degasser, temperature controlled column compartment, sample auto injector and diode array detector. In addition, the Model 1100 has a mass selective detector. The column used was a Hewlitt Packard Zorbax Eclipse XDB-C8, 5 micron, 4.6 mm×150 mm. The flow rate was 1.0 ml per minute, and the column was developed with the gradient shown in Table XI below.

TABLE XI
HPLC Elution Conditions for Analysis of Culture Supernatants

| | % (v/v) of 0.1% (v/v) | % (v/v) of |
|---|---|---|
| Time (minutes) | Phosphoric Acid | Acetonitrile |
| 0.00 | 65 | 35 |
| 5.00 | 65 | 35 |
| 5.50 | 55 | 45 |
| 12.00 | 55 | 45 |
| 15.00 | 65 | 35 |

The eluate was analyzed at 210 nm with a bandwidth of 8 nm.

An initial experiment was conducted to test the stability of Precursor 2. Precursor 2 was dissolved in water and two different culture media; Euglena Medium, and Kratz and Meyers Medium to yield solutions of 100 μg per ml. The solutions were incubated for 3 days at room temperature, and 5 μl of each was injected into the column and compared to a freshly prepared sample of Precursor 2 dissolved in water. Precursor 2 eluted at 10.3 minutes. The recovery of the precursor was virtually quantitative in water, 93.1% in Kratz and Meyers Medium, but 31.1% in Euglena Medium. In the latter case, a single new peak was observed at 2.0 minutes. In addition, similar stability studies were performed on Precursor 2 in water at pH 4 and pH 9. The recovery of the precursor was lowered at pH 4, but not at pH 9.

5.0 μl of a solution containing both Precursor 2 and its putative degradate were injected into an 1100 HPLC equipped with an electrospray interface (API), and the mass spectrums of the parent and unknown peaks were measured. The conditions used for the experiment are listed in Table XII.

TABLE XII
Conditions for Mass Determination
of Putative Carbamate Degradate
HPLC CONDITIONS:

| | |
|---|---|
| Column | Zorbax ODS 10 μm, 4.6 × 150 mm |
| Flow Rate | 1.00 mL/min |
| Injection Volume | 25 μL |
| Mobile Phase | Isocratic |
| Solvent A | 65% 0.1% Trifluoroacetic acid |
| Solvent B | 35% Acetonitrile |

MASS SPECTROSCOPY CONDITIONS

| | |
|---|---|
| Interface | Electrospray |
| Gas Temperature | 350° C. |
| Drying Gas | Nitrogen, 10 L/min |
| Nebulizer Pressure | 25 psig |
| Ionization Voltage | 3500 volts |
| Scan Range | 50-1500 amu |

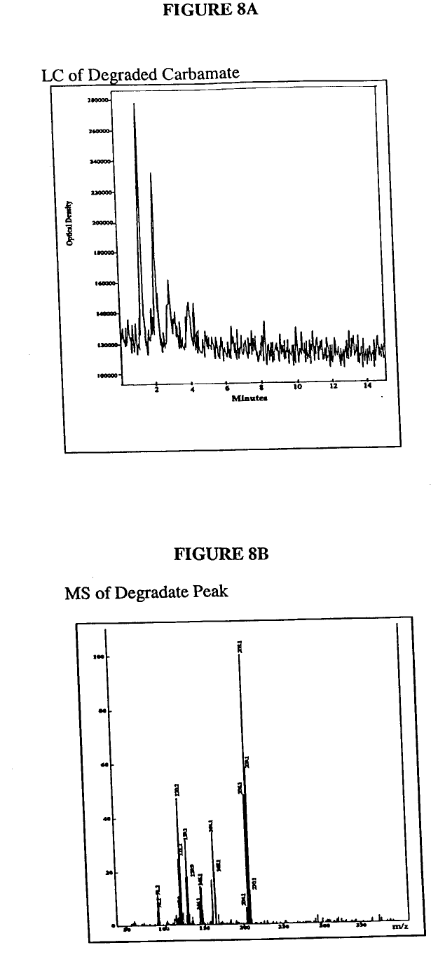

FIG. 8A shows an HPLC-MS chromatograph from a typical experiment, and FIG. 8B shows the positive mode mass spectrum of the peak eluting at 2.1 minutes. The mass of the putative degradate and the masses of its fragments are compatible with an acid-catalyzed hydrolysis of the ester bond of the original parent molecule to produce tertiary butanol and (oxiranyl-2-phenylethyl)carbamic acid. Consistent with this interpretation, it was found that the sum of the total UV absorption from the 10.3 and 2 minute peaks was linear with regard to concentration of starting Precursor 2. It was concluded that the hydrolytic degradation process is a single reaction yielding tertiary butanol and (oxiranyl-2-phenylethyl)carbamic acid. Analysis of HPLC results from biotransformation experiments was interpreted taking into account the possible non-biological hydrolysis of the ester bond.

(2) Purification

In the present example, culture supernatants were not extracted with an organic solvent. It is possible to extract the supernatants by, for example, adding an equal volume of an organic solvent that is not miscible with water, mixing well to emulsify the mixture, and then separating the two liquid phases by centrifugation or in a separatory funnel or by some other means. Examples of suitable organic solvents include but are not limited to benzene and ethyl acetate.

(3) Analysis

Culture supernatants were analyzed on a Hewlett Packard High Pressure Liquid Chromatograph (HPLC), Model 1100 or 1050, equipped with solvent delivery system, solvent degasser, temperature controlled column compartment, sample auto injector and diode array detector. In addition, the Model 1100 has a mass selective detector. The column used was a Hewlitt Packard Zorbax Eclipse XDB-C8, 5 micron, 4.6 mm×150 mm. The flow rate was 1.0 ml per minute. 5.0 μl of extract aliqots were injected and the column was developed with the gradient shown in Table XIII below.

TABLE XIII
HPLC Elution Conditions for Analysis of Culture Supernatants

| | % of 0.1% (v/v) | % (v/v) of |
|---|---|---|
| Time (minutes) | Phosphoric Acid | Acetonitrile |
| 0.00 | 65 | 35 |
| 5.00 | 65 | 35 |
| 5.50 | 55 | 45 |
| 12.00 | 55 | 45 |
| 15.00 | 65 | 35 |

The eluate was analyzed at 210 nm with a bandwidth of 8 nm. Each chromatograph of culture supernatant was compared to chromatograms of culture growth control supernatant and of biotransformation control supernatant. Peaks that appeared in the supernatants of cultures incubated with algae and precursor compound, but not in control samples, were considered candidates for modified Precursor 2 (metabolite), and were selected for further study. Of the 12 strains tested, strains, Bracteacoccus cinnibarinus had two candidate peaks, and Crytomonas ovata exhibited 3 putative metabolite peaks. The elution time for the larger candidate peak from the Bracteacoccus cinnibarinus culture was almost identical to that for one of the candidate peaks in Cryptomonas ovata, possibly indicating identical transformations of the precursor.

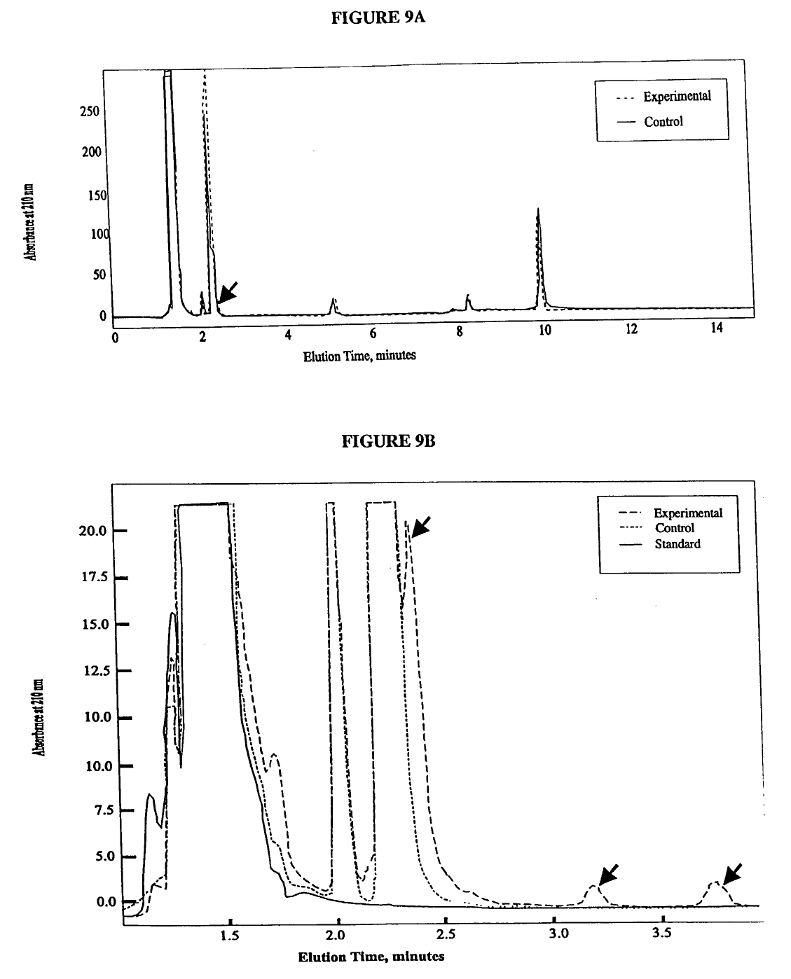

FIG. 9A shows a representative TPLC chromatograph using the culture supernatant from Cryptomonas ovata with the candidate peaks marked. FIG. 9B shows the area of the HPLC chromatogram around one of the marked peaks in FIG. 9A (shown at an increased resolution).

The peak at about 2.4 minutes in FIG. 9B was subjected to further analysis by combined liquid chromatography and mass spectroscopy (LC/MS) using a Phenomenex Luna 3 micron phenyl-hexyl column, 150 mm×2.0 mm. The column was developed at 30° C., at 0.40 ml per minute with the solvent program shown in Table XIV below.

TABLE XIV
LC/MS Elution Conditions for the
Analysis of Candidate HPLC Peaks by MS

| Elapsed | % of 0.1% (v/v) | % (v/v) of | |
|---|---|---|---|
| Time | Formic Acid | Acetonitrile | Curve Type |
| 0.00 | 95 | 5 | — |
| 1.00 | 95 | 5 | Hold |
| 10.00 | 20 | 80 | Linear |
| 12.00 | 20 | 80 | Hold |
| 13.00 | 95 | 5 | Linear |
| 18.00 | 95 | 5 | Re-equilibration |

For mass spectroscopy, the ionization mode was ESP positive, with a cone voltage of 15 or 28 volts. The mass range was 60 to 500 amu, the source temperature was 130° C., and the time window was 0 to 12 minutes. The ESI nebulizer gas was nitrogen at 15 l/hr, and the bath gas was also nitrogen but at 350 l/hr. The mass spectrometer was a Micromass Quattro II equipped with an ESP Z-spray source.

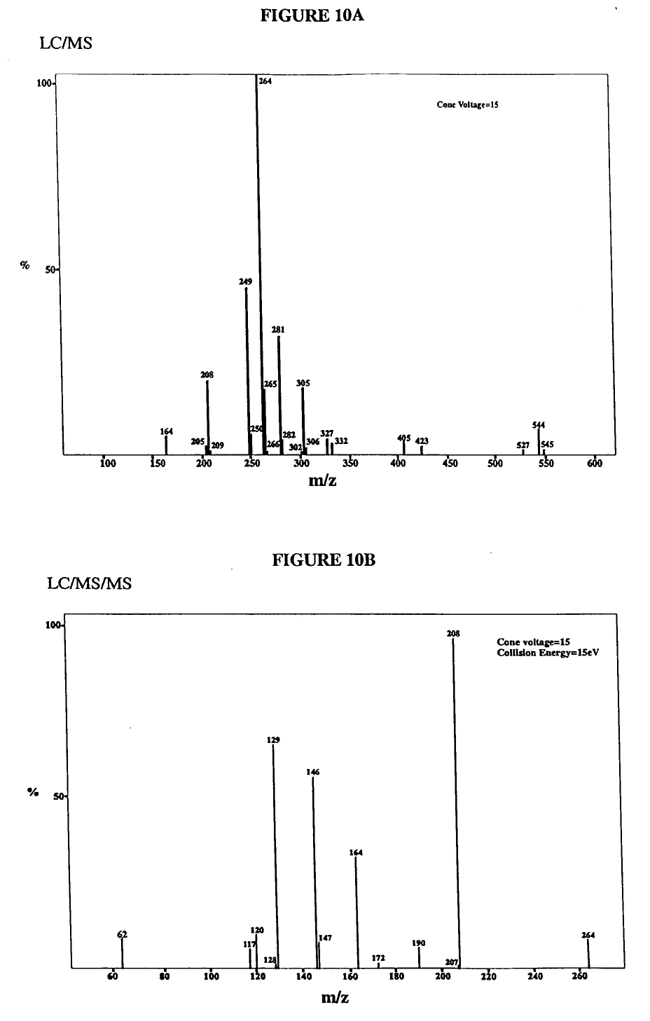

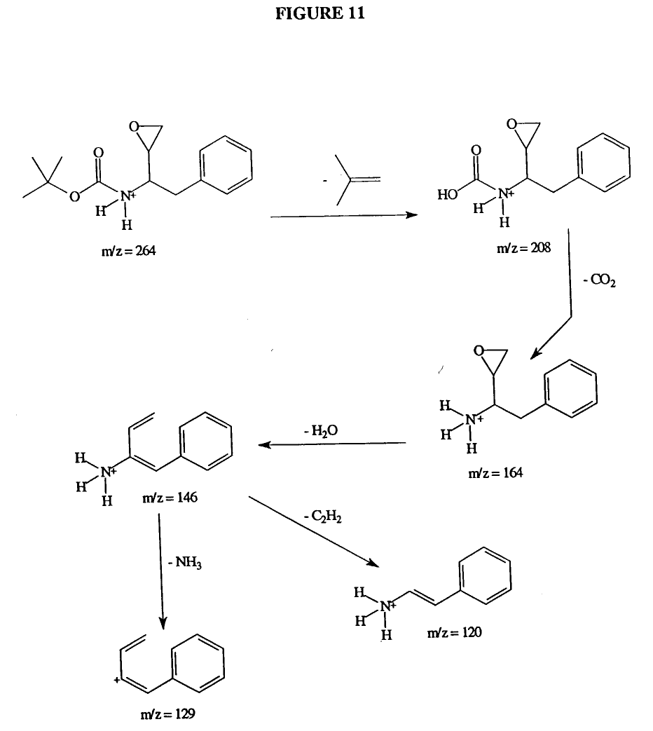

The precursor compound eluted from the LC column at 10.11 minutes. FIG. 10A shows the mass spectrograph of that peak. The parent molecule has a protonated mass of 264 and a mass for the adduct between the protonated parent and acetonitrile of 305. There is a major peak at 281, consistent with hydrolysis of the epoxide ring. In addition, a major peak at 208 is consistent with protonation and the loss of the t-butyl group, and the major peak at 249 is consistent with an acetonitrile adduct with the molecule of the 208 peak. The LC/MS/MS analysis (FIG. 10B) is consistent with the fragmentation scheme shown in FIG. 11.

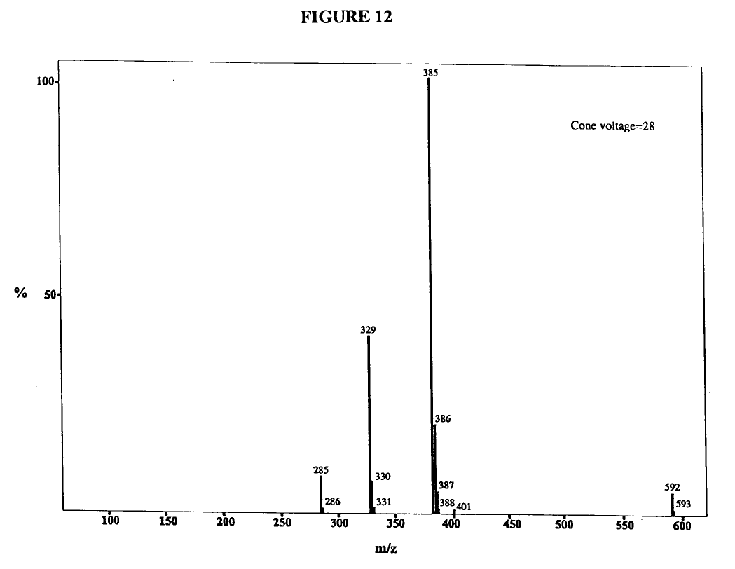

FIG. 12 shows the LC/MS scan for the candidate peak eluting at 7.09 minutes. The candidate peak had two mass components, a major peak at a mass of 385 and a minor peak of mass 592. The latter peak is consistent with a polymerization product between the species of mass 385 and a molecule of mass 207. The parent compound is predicted to decompose via the loss of its tertiary butyl group to a molecular weight of 207, as shown above.

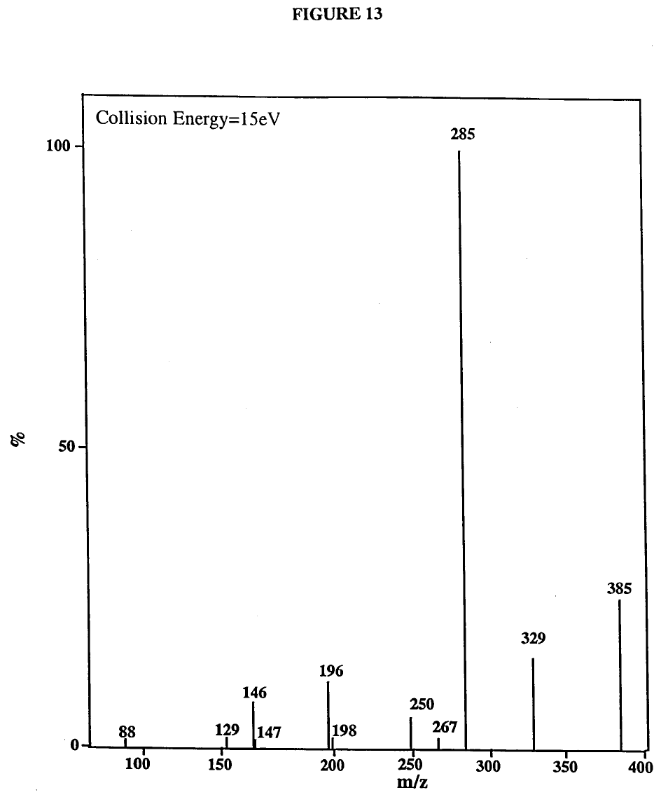

In order to determine the structure of the species of MW 385, liquid chromatography coupled to 2-dimensional mass spectroscopy (LC/MS/MS) was performed. LC/MS/MS was performed under the same conditions as LC/MS, except that the collision gas was argon and the collision energy was 15 or 25 eV. The results were analyzed using MassLynx Version 3.4 (Micromass) software. FIG. 13 shows the LC/MS/MS scan for m/z 385.

The unknown appears to have a molecular weight of 384 with a protonated mass of 385. The distribution of the protonated molecular ion between m/z 385, 386, and 387 is consistent with the presence of one sulfur atom. Molecular modeling based on the natural abundances of C12, C13, S32, and S34 predicts a peak height for m/z 386 that is 22% of m/z 385, and for m/z 387 that is 8.72% of m/z 385. The actual values of 21% and 8.9% agree closely with this model. In addition, ions at m/z 329 and 285 indicate that a 1-butyl group is present, attached to a carboxyl group. Furthermore, the even molecular weight indicates that there is an even number of nitrogen atoms. The parent molecule had one nitrogen, so it was concluded that the Cryptomonas algae constructed an adduct between the parent molecule and a group containing one sulfur, a mass of 121, and an odd number of nitrogens, probably one due to the mass constraint. The most likely candidate is cysteine. The fragmentation pattern suggests that the epoxide ring in the parent molecule was opened, adding a hydroxyl group. The addition of a hydroxyl at the epoxide ring suggests that the adduct between cysteine and Precursor 2 was a dehydration reaction.

To further elucidate the structure of the metabolite, quantitative time of flight mass spectroscopy was performed using the Q-T of micro™ system (Micromass). Electrospray LC/MS was performed using a Phenomenex 3 micron phenyl-hexyl column, 100 mm×4.6 mm. The column was developed at a flow rate of 0.35 ml per minute with the solvent program shown in Table XV below.

TABLE XV
Q-Tof micro LC/MS Elution Conditions for the
Analysis of Candidate HPLC Peak by MS

| | % of 95% | | |
|---|---|---|---|
| | H₂0, 5% | | |
| | Acetonitrile, | | |
| Elapsed | 1% Formic | % (v/v) of | |
| Time | Acid | Acetonitrile | Curve Type |
| 0.00 | 95 | 5 | — |
| 1.00 | 95 | 5 | Hold |
| 15.00 | 20 | 80 | Linear |
| 15.00 | 20 | 80 | Hold |
| 20.00 | 95 | 5 | Reequilibrate |

For mass spectroscopy, the ionization mode was positive ion, with a cone voltage of 15V. The source temperature was 135° C., the desolvation temperature was 325° C., and the nebulizer gas was argon at 17 psi. The 385 MW metabolite eluted from the column at 11.8 minutes, and was measured to have a more precise mass of 385.1788, corresponding within 0.9 milliDaltons of the molecular formula C₁₈H₂₉N₂O₅S. This is consistent with the interpretation of the quadropole LC/MS as discussed above.

The 385 MW metabolite was further subjected to LC/S/MS using the Q-T of micro™ system. Conditions were identical to those used for the Q-T of LC/MS except that the collision energy was either 15V or 25V. Molecular masses and likely formulae of the major fragment produced from the 385 MW metabolite are listed in Table XVI below. In all cases, the two different collision energies produced similar actual masses and probable formulae.

TABLE XVI
Major Fragments of MW 385 Metabolite

| | Most Consistent |
|---|---|
| Molecular Mass | Molecular Formula |
| 329.117 | C14H21N2O5S |
| 250.090 | C13H16NO2S |
| 196.080 | C10H14NOS |
| 161.045 | C10H9S |
| 146.094 | C10H12N |
| 129.073 | C7H13S |

Two possible structures are consistent with the results, N1-(tert-butoxycarbonyl)-N-1-[1-phenyl(2,3-dihydroxypropyl)methyl]cysteinamide and S-{2-hydroxy-3-[(tert-butoxycarbonyl)amino]-4-phenylbutyl}cysteine.

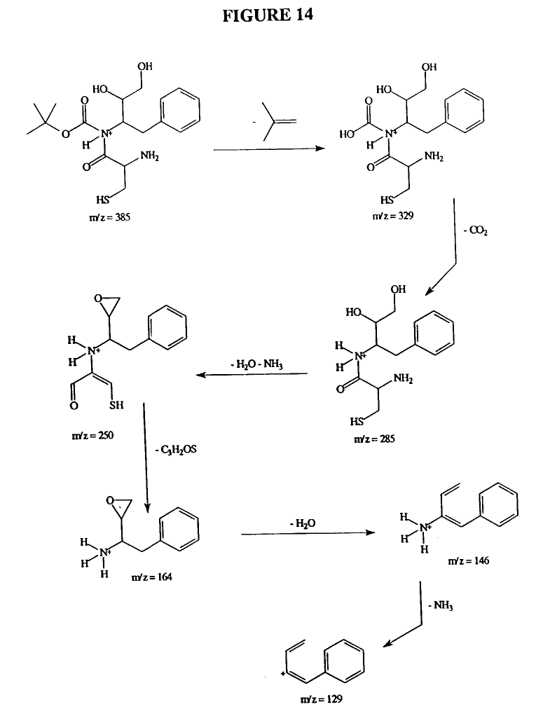

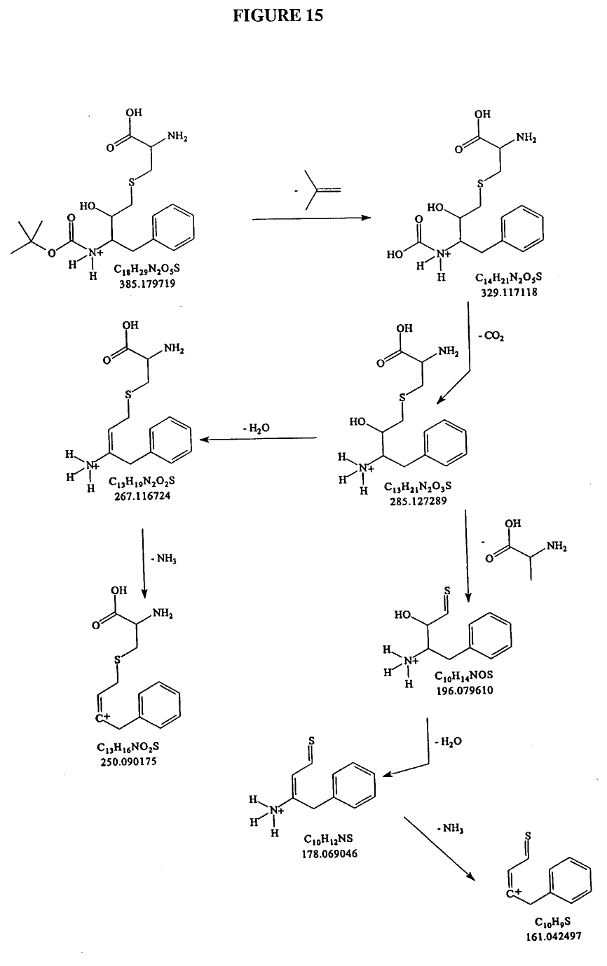

The daughter peaks are consistent with the fragmentation patterns of the hypothetical molecules shown in FIG. 14 and FIG. 15, respectively.

While the invention has been described in detail and with reference to specific embodiments thereof, it will be apparent to one skilled in the art that various changes and modifications can be made therein without departing from the spirit and scope thereof.

What is claimed:

1. A process for biotransformation of a precursor compound comprising:
(A) obtaining a panel of different non-prokaryotic microalgae which are evolutionarily and ecologically diverse;
(B) exposing each member of said panel of non-prokaryotic microalgae to a solvent for the precursor compound, and selecting a subset of non-prokaryotic microalgae that grow in the presence of said solvent;
(C) exposing each non-prokaryotic microalgae of the resulting subset to the precursor compound, and selecting a further subset of non-prokaryotic microalgae whose growth is inhibited or increased in the presence of said precursor compound;
(D) growing the resulting further subset of non-prokaryotic microalgae in the presence of said precursor compound so as to transform said precursor compound to produce a metabolite of said precursor compound, and so as to obtain a cellular biomass of said non-prokaryotic microalgae and a culture supernatant;
(E) separating the resulting cellular biomass from the resulting culture supernatant and optionally extracting the resulting separated cell biomass with said solvent so as to obtain said metabolite of said precursor compound; and optionally
(F) purifying said metabolite from the resulting culture supernatant or solvent extract of the resultant biomass and analyzing said metabolite so as to identify the structure of said metabolite and the modification in said precursor compound.

2. The process of claim 1, wherein said panel consists of non-prokaryotic microalgae selected from at least one member of the group consisting of Charophyta, Chlorophyta, Diatoms, Rhodophyta, Cryptophyta, Chlorarachniophyta, Haptophyta, Euglenophyta and Heterokonta.

3. The process of claim 2, wherein said Charophyta is selected from at least one member the group consisting of Zygenematophyceae, Mesostigmatophyceae, Chlorokybophyceae, Coleochaetophyceae and Klebsormidiophyceae.

4. The process of claim 3, wherein said Zygenematophyceae is selected from at least one member the group consisting of Actinotaenium, Arthrodesmus, Bambusina, Closterium, Cosmarium, Cosmocladium, Desmidium, Euastrum, Genicularia, Gonatozygon, Heimansia, Hyalotheca, Mesotaenium, Micrasterias, Mougeotia; Netrium, Onychonema, Penium, Phymatodocis, Pleurotaenium, Roya, Sphaerozosma, Spirogyra, Spondylosium, Staurastrum, Staurodesmus, Teilingia, Triploceras, Xanthidium, Zygnema and Zygogonium.

5. The process of claim 3, wherein said Mesostigmatophyceae is selected from at least one member the group consisting of Chaetosphaeridium and Mesotstigma.

6. The process of claim 3, wherein said Chlorokybophyceae is Chlorokybus.

7. The process of claim 3, wherein said Coleochaetophyceae is Coleochaete.

8. The process of claim 3, wherein said Klebsormidiophyceae is Klebsormidium.

9. The process of claim 2, wherein said Chlorophyta is selected from at least one member the group consisting of Chlorophyceae, Ulvophyceae, Trebouxiophyceae, Prasiniophyceae and Charophyceans.

10. The process of claim 9, wherein said Chlorophyceae is selected from at least one member of the group consisting of Acetabularia, Acicularia, Actinochloris, Amphikrikos, Anadyomene, Ankistrodesmus, Ankyra, Aphanochaete, Ascochloris, Asterococcus, Asteromonas, Astrephomene, Atractomorpha, Axilococcus, Axilosphaera, Basichlamys, Basicladia, Binuclearia, Bipedinomonas, Blastophysa, Boergesenia, Boodlea, Borodinella, Borodinellopsis, Botryococcus, Brachiomonas, Bracteacoccus, Bulbochaete, Caespitella, Capsosiphon, Carteria, Centrosphaera, Chaetomorpha, Chaetonema, Chaetopeltis, Chaetophora, Chalmasia, Chamaetrichon, Characiochloris, Characiosiphon, Characium, Chlamydella, Chlamydobotrys, Chlamydocapsa, Chlamydomonas, Chlamydopodium, Chloranomala, Chlorochydridion, Chlorochytrium, Chlorocladus, Chlorocloster, Chlorococcopsis, Chlorococcum, Chlorogonium, Chloromonas, Chlorophysalis, Chlorosarcina, Chlorosarcinopsis, Chlorosphaera, Chlorosphaeropsis, Chlorotetraedron, Chlorothecium, Chodatella, Choricystis, Cladophora, Cladophoropsis, Cloniophora, Closteriopsis, Coccobotrys, Coelastrella, Coelastropsis, Coelastrum, Coenochloris, Coleochlamys, Coronastrum, Crucigenia, Crucigeniella, Ctenocladus, Cylindrocapsa, Cylindrocapsopsis, Cylindrocystis, Cymopolia, Cystococcus, Cystomonas, Dactylococcus, Dasycladus, Deasonia, Derbesia, Desmatractum, Desmodesmus, Desmotetra, Diacanthos, Dicellula, Dicloster, Dicranochaete, Dictyochloris, Dictyococcus, Dictyosphaeria, Dictyosphaerium, Didymocystis, Didymogenes, Dilabifilum, Dimorphococcus, Diplosphaera, Draparnaldia, Dunaliella, Dysmorphococcus, Echinocoleum, Elakatothrix, Enallax, Entocladia, Entransia, Eremosphaera, Ettlia, Eudorina, Fasciculochloris, Fernandinella, Follicularia, Fottea, Franceia, Friedmannia, Fritschiella, Fusola, Geminella, Gloeococcus, Gloeocystis, Gloeodendron, Gloeomonas, Gloeotila, Golenkinia, Gongrosira, Gonium, Graesiella, Granulocystis, Gyorffiana, Haematococcus, Hazenia, Helicodictyon, Hemichloris, Heterochlamydomonas, Heteromastix, Heterotetracystis, Hormidiospora, Hormidium, Hormotila, Hormotilopsis, Hyalococcus, Hyalodiscus, Hyalogonium, Hyaloraphidium, Hydrodictyon, Hypnomonas, Ignatius, Interfilum, Kentrosphaera, Keratococcus, Kermatia, Kirchneriella, Koliella, Lagerheimia, Lautosphaeria, Leptosiropsis, Lobocystis, Lobomonas, Lola, Macrochloris, Marvania, Micractinium, Microdictyon, Microspora, Monoraphidium, Muriella, Mychonastes, Nanochlorum, Nautococcus, Neglectella, Neochloris, Neodesmus, Neomeris, Neospongiococcum, Nephrochlamys, Nephrocytium, Nephrodiella, Oedocladium, Oedogonium, Oocystella, Oocystis, Oonephris, Ourococcus, Pachycladella, Palmella, Palmellococcus, Palmellopsis, Palmodictyon, Pandorina, Paradoxia, Parietochloris, Pascherina, Paulschulzia, Pectodictyon, Pediastrum, Pedinomonas, Pedinopera, Percursaria, Phacotus, Phaeophila, Physocytium, Pilina, Planctonema, Planktosphaeria, Platydorina, Platymonas, Pleodorina, Pleurastrum, Pleurococcus, Ploeotila, Polyedriopsis, Polyphysa, Polytoma, Polytomella, Prasinocladus, Prasiococcus, Protoderma, Protosiphon, Pseudendocloniopsis, Pseudocharacium, Pseudochlorella, Pseudochlorococcum, Pseudococcomyxa, Pseudodictyosphaerium, Pseudodidymocystis, Pseudokirchneriella, Pseudopleurococcus, Pseudoschizomeris, Pseudoschroederia, Pseudostichococcus, Pseudotetracystis, Pseudotetraëdron, Pseudotrebouxia, Pteromonas, Pulchrasphaera, Pyramimonas, Pyrobotrys, Quadrigula, Radiofilum, Radiosphaera, Raphidocelis, Raphidonema, Raphidonemopsis, Rhizoclonium, Rhopalosolen, Saprochaete, Scenedesmus, Schizochlamys, Schizomeris, Schroederia, Schroederiella, Scotiellopsis, Siderocystopsis, Siphonocladus, Sirogonium, Sorastrum, Spermatozopsis, Sphaerella, Sphaerellocystis, Sphaerellopsis, Sphaerocystis, Sphaeroplea, Spirotaenia, Spongiochloris, Spongiococcum, Stephanoptera, Stephanosphaera, Stigeoclonium, Struvea, Tetmemorus, Tetrabaena, Tetracystis, Tetradesmus, Tetraedron, Tetrallantos, Tetraselmis, Tetraspora, Tetrastrum, Treubaria, Triploceros, Trochiscia, Trochisciopsis, Ulva, Uronema, Valonia, Valoniopsis, Ventricaria, Viridiella, Vitreochlamys, Volvox, Volvulina, Westella, Willea, Wislouchiella, Zoochlorella, Zygnemopsis, Hyalotheca, Chlorella, Pseudopleurococcum and Rhopalocystis.

11. The process of claim 9, wherein said Ulvophyceae is selected from at least one member the group consisting of Acrochaete, Bryopsis, Cephaleuros, Chlorocystis, Enteromorpha, Gloeotilopsis, Halochlorococcum, Ostreobium, Pirula, Pithophora, Planophila, Pseudendoclonium, Trentepohlia, Trichosarcina, Ulothrix, Bolbocoleon, Chaetosiphon, Eugomontia, Oltmannsiellopsis, Pringsheimiella, Pseudodendroclonium, Pseudulvella, Sporocladopsis, Urospora and Wittrockiella.

12. The process of claim 9, wherein said Trebouxiophyceae is selected from at least one member the group consisting of Apatococcus, Asterochloris, Auxenochlorella, Chlorella, Coccomyxa, Desmococcus, Dictyochloropsis, Elliptochloris, Jaagiella, Leptosira, Lobococcus, Makinoella, Microthamnion, Myrmecia, Nannochloris, Oocystis, Prasiola, Prasiolopsis, Prototheca, Stichococcus, Tetrachlorella, Trebouxia, Trichophilus, Watanabea and Myrmecia.

13. The process of claim 9, wherein said Prasiniophyceae is selected from at least one member the group consisting of Bathycoccus, Mantoniella, Micromonas, Nephroselmis, Pseudoscourfieldia, Scherffelia, Picocystis, Pterosperma and Pycnococcus.

14. The process of claim 9, wherein said Charophyceans is Zygogonium.

15. The process of claim 2, wherein said Diatoms is selected from at least one member of the group consisting of Bolidophyceae, Coscinodiscophyceae, Dinophyceae and Alveolates.

16. The process of claim 15, wherein said Bolidophyceae is selected from at least one member of the group consisting of Bolidomonas, Chrysophyceae, Giraudyopsis, Glossomastix, Chromophyton, Chrysamoeba, Chrysochaete, Chrysodidymus, Chrysolepidomonas, Chrysosaccus, Chrysosphaera, Chrysoxys, Cyclonexis, Dinobryon, Epichrysis, Epipyxis, Hibberdia, Lagynion, Lepochromulina, Monas, Monochrysis, Paraphysomonas, Phaeoplaca, Phaeoschizochlamys, Picophagus, Pleurochrysis, Stichogloea and Uroglena.

17. The process of claim 15, wherein said Coscinodiscophyceae is selected from the group consisting of Bacteriastrum, Bellerochea, Biddulphia, Brockmanniella, Corethron, Coscinodiscus, Eucampia, Extubocellulus, Guinardia, Helicotheca, Leptocylindrus, Leyanella, Lithodesmium, Melosira, Minidiscus, Odontella, Planktoniella, Porosira, Proboscia, Rhizosolenia, Stellarima, Thalassionema, Bicosoecid, Symbiomonas, Actinocyclus, Amphora, Arcocellulus, Detonula, Diatoma, Ditylum, Fragilariophyceae, Asterionellopsis, Delphineis, Grammatophora, Nanofrustulum, Synedra and Tabularia.

18. The process of claim 15, wherein said Dinophyceae is selected from at least one member of the group consisting of Adenoides, Alexandrium, Amphidinium, Ceratium, Ceratocorys, Coolia, Crypthecodinium, Exuviaella, Gambierdiscus, Gonyaulax, Gymnodinium, Gyrodinium, Heterocapsa, Katodinium, Lingulodinium, Pfiesteria, Polarella, Protoceratium, Pyrocystis, Scrippsiella, Symbiodinium, Thecadinium, Thoracosphaera and Zooxanthella.

19. The process of claim 15, wherein said Alveolates is selected from at least one member of the group consisting of Cystodinium, Glenodinium, Oxyrrhis, Peridinium, Prorocentrum and Woloszynskia.

20. The process of claim 2, wherein said Rhodophyta is selected from at least one member of the group consisting of Acrochaetium, Agardhiella, Antithamnion, Antithamnionella, Asterocytis, Audouinella, Balbiania, Bangia, Batrachospermum, Bonnemaisonia, Bostrychia, Callithamnion, Caloglossa, Ceramium, Champia, Chroodactylon, Chroothece, Compsopogon, Compsopogonopsis, Cumagloia, Cyanidium, Cystoclonium, Dasya, Digenia, Dixoniella, Erythrocladia, Erythrolobas, Erythrotrichia, Flintiella, Galdieria, Gelidium, Glaucosphaera, Goniotrichum, Gracilaria, Grateloupia, Griffithsia, Hildenbrandia, Hymenocladiopsis, Hypnea, Laingia, Membranoptera, Myriogramme, Nemalion, Nemnalionopsis, Neoagardhiella, Palmaria, Phyllophora, Polyneura, Polysiphonia, Porphyra, Porphyridium, Pseudochantransia, Pterocladia, Pugetia, Rhodella, Rhodochaete, Rhodochorton, Rhodosorus, Rhodospora, Rhodymenia, Seirospora, Selenastrum, Sirodotia, Solieria, Spermothamnion, Spyridia, Stylonema, Thorea, Trailiella and Tuomeya.

21. The process of claim 2, wherein said Cryptophyta is selected from at least one member of the group consisting of Campylomonas, Chilomonas, Chroomonas, Cryptochrysis, Cryptomonas, Goniomonas, Guillardia, Hanusia, Hemiselmis, Plagioselmis, Proteomonas, Pyrenomonas, Rhodomonas and Stroreatula.

22. The process of claim 2, wherein said Chlorarachniophyta is selected from at least one member of the group consisting of Chlorarachnion, Lotharella and Chattonella.

23. The process of claim 2, wherein said Haptophyta is selected from at least one member of the group consisting of Pavlovophyceae and Prymnesiophyceae.

24. The process of claim 21, wherein said Pavlovophyceae is selected from at least one member of the group consisting of Apistonema, Chrysochromulina, Coccolithophora, Corcontochrysis, Cricosphaera, Diacronema, Emiliana, Pavlova and Ruttnera.

25. The process of claim 21, wherein said Prymnesiophyceae is selected from at least one member of the group consisting of Cruciplacolithus, Prymnesium, Isochrysis, Calyptrosphaera, Chrysotila, Coccolithus, Dicrateria, Heterosigma, Hymenomonas, Imantonia, Gephyrocapsa, Ochrosphaera, Phaeocystis, Platychrysis, Pseudoisochrysis, Syracosphaera and Pleurochrysis.

26. The process of claim 2, where said Euglenophyta is selected from at least one member of the group consisting of Astasia, Colacium, Cyclidiopsis, Distigma, Euglena, Eutreptia, Eutreptiella, Gyropaigne, Hyalophacus, Khawkinea Astasia, Lepocinclis, Menoidium, Parmidium, Phacus, Rhabdomonas, Rhabdospira, Tetruetreptia and Trachelomonas.

27. The process of claim 2, wherein said Heterokonta is selected from at least one member of the group consisting of Phaeophyceae, Pelagophyceae, Xanthophyceae, Eustigmatophyceae, Syanurophyceae, Phaeothamniophyceae and Raphidophyceae.

28. The process of claim 27, wherein said Phaeophyceae is selected from at least one member of the group consisting of Ascoseira, Asterocladon, Bodanella, Desmarestia, Dictyocha, Dictyota, Ectocarpus, Halopteris, Heribaudiella, Pleurocladia, Porterinema, Pylaiella, Sorocarpus, Spermatochnus, Sphacelaria and Waerniella.

29. The process of claim 27, wherein said Pelagophyceae is selected from at least one member of the group consisting of Aureococcus, Aureoumbra, Pelagococcus, Pelagomonas, Pulvinaria and Sarcinochrysis.

30. The process of claim 27, wherein said Xanthophyceae is selected from at least one member of the group consisting of Asterosiphon, Botrydiopsis, Botrydium, Bumilleria, Bumilleriopsis, Characiopsis, Chlorellidium, Chlorobotrys, Goniochloris, Heterococcus, Heterothrix, Heterotrichella, Mischococcus, Ophiocytium, Pleurochloridella, Pleurochloris, Pseudobumilleriopsis, Sphaerosorus, Tribonema, Vaucheria and Xanthonema.

31. The process of claim 27, wherein said Eustigmatophyceae is selected from at least one member of the group consisting of Chloridella, Ellipsoidion, Eustigmatos, Monodopsis, Monodus, Nannochloropsis, Polyedriella, Pseudocharaciopsis, Pseudostaurastrum and Vischeria.

32. The process of claim 27, wherein said Syanurophyceae is selected from at least one member of the group consisting of Mallomonas, Synura and Tessellaria.

33. The process of claim 27, wherein said Phaeothamniophyceae is selected from at least one member of the group consisting of Phaeobotrys and Phaeothamnion.

34. The process of claim 27, wherein said Raphidophyceae is selected from at least one member of the group consisting of Olisthodiscus, Vacuolaria and Fibrocapsa.

35. The process of claim 1, wherein said precursor compound is a racemic mixture and said metabolite is a chirally pure derivative of one enantiomer of said precursor compound.

36. A method for obtaining a metabolite of a precursor compound comprising:
(A) culturing a member of the further subset of non-prokaryotic microalgae obtained in step (C) of claim 1 in the presence of said precursor compound, or
(B) contacting a cell extract of said member or enzymes purified therefrom with said precursour compound, and
purifying the resulting metabolite from the culture supernatant or biomass, or said cell extract or enzymes.

37. A method comprising contacting a mammal with the metabolite obtained by the process of claim 36, and assaying for toxicity of said metabolite in said mammal, wherein said precursor compound is a pharmaceutical, food additive or hazardous waste.

38. The method of claim 1, wherein said precursor compound is a heterocyclic compound whose heterocyclic ring contains 2-7 carbon atoms and 1-3 heteroatoms each selected from the group consisting of oxygen, sulfur and nitrogen.

39. The method of claim 1, wherein said precursor compound is a heterocyclic compound whose heterocyclic ring contains 2 carbon atoms and 1 heteroatom selected from the group consisting of oxygen and nitrogen.

40. The method of claim 38, wherein said heterocyclic compound is an oxazolidine represented by 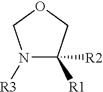
wherein R¹, R² and R³ are each independently selected from the group consisting of hydrogen, hydroxyl, halogen, optionally substituted amino, optionally substituted nitro, optionally substituted sulfo, optionally substituted phospho, optionally substituted alkyl (C₁₋₂₀), optionally substituted cycloaliphatic (C₁₋₂₀), optionally substituted aromatic (C₅₋₂₀), and optionally substituted heterocyclic (C₃₋₂₀) groups.

41. The method of claim 1, wherein said precursor compound is a heterochain compound whose backbone consists of 4-12 carbon atoms and 1-3 heteroatoms selected from the group consisting of nitrogen, oxygen, phosphorus or sulfur.

42. The method of claim 41, wherein said heterochain compound is an N-substituted aminide represented by 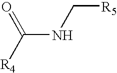
wherein R₄, and R₅ are each independently selected from the group consisting of hydrogen, hydroxyl, halogen, optionally substituted amino, optionally substituted nitro, optionally substituted sulfo, optionally substituted phospho, optionally substituted alkyl (C₁₋₂₀), optionally substituted cycloaliphatic (C₁₋₂₀), optionally substituted aromatic (C₅₋₂₀), and optionally substituted heterocyclic (C₃₋₂₀) groups.

43. N1-(tert-butoxycarbonyl)-N1-[1-phenyl(2,3-dihydroxypropyl)methyl]cysteinamide.

44. S-{2-hydroxy-3-[(tert-butoxycarbonyl)amino]-4-phenylbutyl}cysteine.

45. A method for preparing N1-(tert-butoxycarbonyl)-N-1-[1-phenyl(2,3-dihydroxypropyl)methyl]cysteinamide, comprising:
(A) culturing Cryptomonas, in the presence of tert-butyl[S-(R*-R*)]-(−)-(1-oxiranyl-2-phenylethylcarbamate); or
(B) contacting a cell extract of Cryptomonas or enzymes purified therefrom with tert-butyl[S-(R*-R*)]-(−)-(1-oxiranyl-2-phenylethylcarbamate),
so as to produce N1-(tert-butoxycarbonyl)-N1-[1-phenyl(2,3-dihydroxypropyl)methyl]cysteinamide.

46. The method of claim 45, wherein said Cryptomonas is Cryptomonas ovata.

47. A method for preparing S-{2-hydroxy-3-[(tert-butoxycarbonyl)amino]-4-phenylbutyl}cysteine, comprising:
(A) culturing Cryptomonas in the presence of tert-butyl[S-(R*-R*)]-(−)-(1-oxiranyl-2-phenylethylcarbamate); or
(B) contacting a cell extract of Cryptomonas or enzymes purified therefrom with tert-butyl[S-(R*-R*)]-(−)-(1-oxiranyl-2-phenylethylcarbamate),
so as to produce S-{2-hydroxy-3-[(tert-butoxycarbonyl)amino]-4-phenylbutyl}cysteine.

48. The method of claim 47, wherein said Cryptomonas is Cryptomonas ovata.

49. A method for preparing (S)-(−)-3-(Benzyloxycarbonyl)-1-amino-2-hydroxycarboxylic acid, comprising:
(A) culturing Chlamydomonas in the presence of (S)-(−)-3-(Benzyloxycarbonyl)-4-oxazolidinecarboxylic acid; or
(B) contacting a cell extract of Chlamydomonas or enzymes purified therefrom with (S)-(−)-3-(Benzyloxycarbonyl)-4-oxazolidinecarboxylic acid,
so as to produce (S)-(−)-3-(Benzyloxycarbonyl)-4-oxazolidinecarboxylic acid.

50. The method of claim 49, wherein said Chlamydomonas is Chlamydomonas reinhardtii.

51. A method for preparing S-(2-hydroxyethyl) cysteine or a derivative thereof containing substitutions in the ethyl group, comprising:
(A) culturing Cryptomonas in the presence of oxirane or a 2-substituted derivative thereof, or
(B) contacting a cell extract of Cryptomonas or enzymes purified therefrom with 2-oxirane or a 2-substituted derivative thereof,
so as to produce S-(2-hydroxyethyl)cysteine or a derivative thereof containing substitutions in the ethyl group.

52. The method of claim 51, wherein said Cryptomonas is Cryptomonas ovata.
